(19) United States
(12) Patent Application Publication
(10) Pub. No.: US 2008/0153845 A1
(43) Pub. Date: Jun. 26, 2008

(54) TRPV1 ANTAGONISTS AND USES THEREOF

(75) Inventors: R. Kyle PALMER, Cranbury, NJ (US); Daniel Long, Philadelphia, PA (US); Heather Devantier, Philadelphia, PA (US); S. Paul Lee, Newtown, PA (US); M.N. Tulu Buber, Newtown, PA (US); Robert Bryant, Princeton, NJ (US)

Correspondence Address: STERNE, KESSLER, GOLDSTEIN & FOX P.L.L.C., 1100 NEW YORK AVENUE, N.W., WASHINGTON, DC, 20005, US

(73) Assignee: REDPOINT BIO CORPORATION, Ewing, NJ (US)

(21) Appl. No.: 11/926,911

(22) Filed: Oct. 29, 2007

Related U.S. Application Data
(60) Provisional application No. 60/854,678, filed on Oct. 27, 2006.

(51) Int. Cl. A61K 31/497; A61P 27/00

(52) U.S. Cl. 514/253.11

(57) ABSTRACT

The present invention is directed to the use of TRPV1 antagonists as inhibitors of certain taste perceptions and functions. The invention is also directed to, among other things, compositions comprising the TRPV1 antagonists that can be used in pharmaceutical, food, and other products to inhibit certain taste functions and perceptions.

### CROSS REFERENCE TO RELATED APPLICATIONS

This application claims the benefit of U.S. Prov. Appl. No. 60/854,678, filed Oct. 27, 2006, which is herein incorporated by reference in its entirety.

### BACKGROUND OF THE INVENTION

1. Field of the Invention

The present invention relates to the use of compounds useful as inhibitors of taste functions and perceptions and related uses. The invention is also directed to, among other things, compositions comprising compounds that can be used in pharmaceuticals, food, and other products to inhibit certain taste functions and perceptions.

2. Background Art

Taste perception plays a critical role in both the nutritional status of human beings and the basic survival of animals. Margolskee, R. F., J. Biol. Chem. 277:1-4 (2002); Avenet, P. et al., J Membrane Biol. 112:1-8 (1989). The task of taste perception is generally carried out by taste receptor cells (TRCs). TRCs have the ability to perceive the multitude of compounds that are associated with a given taste and then convert that perception to a signal that is deciphered by the brain, resulting in the sensation of sweet, bitter, sour, salty, or umami (savory) taste. Other taste responses, such as pungent, spicy, and hot/peppery tastes, are thought to be mediated by TRCs and other chemosensory cell types in the tongue.

TRCs are polarized epithelial cells, meaning that they have specialized apical and basolateral membranes. A taste bud contains approximately 60 to 100 TRCs. Each TRC has a portion of its membrane exposed on the mucosal surface of the tongue. Kinnamon, S. C., TINS 11:491-496 (1988). Sensory transduction is initiated by sapid molecules, or “tastants,” that interact with microvillar processes on the apical membrane of TRCs. The tastants bind specific membrane receptors, resulting in a voltage change across the cell membrane. In turn, this depolarizes, or changes the electric potential, of the cell, causing transmitter release and excitation of primary gustatory nerve fibers.

One such membrane receptor is TRPV1, a member of the transient receptor potential (TRP) family of ion channels. TRPV1 forms a channel through the membrane of chemosensory cells and is believed to be activated by a variety of stimuli not limited to heat (>42° C.), protons, endogenous lipoxygenase products, fatty acid amides and a wide range of naturally-derived small molecules such as capsaicin, gingerols, eugenol, piperine and others. See Roberts et al., Rec. Pat. CNS Drug Disc. 1:65-76 (2006); Yang et al., J. Dent. Res. 82:781-785 (2003); Dedov et al., Brit. J. Pharmacol. 137:793-798 (2002); McNamara et al., Brit. J. Pharmacol. 144:781-790 (2005).

Because TRPV1 is a part of the taste-perception machinery, its inhibition prevents an animal from sensing particular tastes. Although taste perception is a vital function, the inhibition of undesirable tastes is beneficial under certain circumstances. For example, many active pharmaceutical ingredients of medicines produce undesirable tastes, such as a bitter or other aversive tastes. Inhibition of these unpalatable tastes produced by the medicine may lead to improved acceptance by the patient.

Traditionally, sweeteners and flavorants have been used to mask one or more aversive tastes of pharmaceuticals. The sweetener or flavorant is known to activate other taste pathways and at sufficiently high concentration this serves to mask an aversive taste of the pharmaceutical. However, this approach has proved ineffective at masking the taste of very unpleasant-tasting compounds. Microencapsulation in a cellulose derivative has also been used to mask the bitter taste of pharmaceuticals. However, this approach prevents rapid oral absorption of the pharmaceutical.

A number of other methods have been suggested to inhibit, alter, or mask unwanted tastes, including the use of 5′-adenosine carboxylic acid (AMP) and 5′-inosine carboxylic acid (IMP) as potential bitterness inhibitors. See U.S. Pat. No. 6,540,978.

Another aspect of taste is its role in food intake. Studies have shown increased food intake as palatability increased. See Sorensen, et al., Int. J. Obes. Relat. Metab. Disord. 27(10):1152-66 (2003). For instance, certain drugs, such as antihypertensives and antihyperlipidemic, have been reported to produce untoward alterations in taste and may result in decreased food intake. See Doty, et al., J Hypertens. 21(10):1805-13 (2003). Taste impairment has also been associated with radiation treatments for head and neck cancer and this taste impairment has been considered to be one of the factors associated with reduced appetite and altered patterns of food intake. See Vissink, et al., Crit. Rev. Oral Biol. Med. 14(3):213-25 (2003). Decreased food consumption has also been correlated with loss of taste sensations in the elderly. See Shiffinan, S. S., J. Am. Med. As'n 278(16):1357-1362 (1997).

There exists a need for compounds that can effectively inhibit an unwanted taste without exhibiting one or more of the side effects of the prior art taste masking agents.

### BRIEF SUMMARY OF THE INVENTION

A first aspect of the present invention is directed to a method of inhibiting an unpleasant taste, said method comprising administering to a subject in need of said inhibition an effective amount of a TRPV1 antagonist or a physiologically acceptable salt thereof.

An additional aspect of the present invention is directed to a method of inhibiting the taste of a pharmaceutical, comprising administering one or more TRPV1 antagonists, or a physiologically acceptable salt thereof, in conjunction with the administration of said pharmaceutical to a subject.

An additional aspect of the present invention is directed to a method of inhibiting the taste of a food product, comprising administering one or more TRPV1 antagonists, or a physiologically acceptable salt thereof, in conjunction with the administration of said food product to a subject.

An additional aspect of the present invention is directed to a method of inhibiting the depolarization of a taste receptor cell or a cell mediating an oral chemosensory response, said method comprising contacting said cell with a TRPV1 antagonist or a physiologically acceptable salt thereof.

An additional aspect of the present invention is directed to a pharmaceutical composition comprising an active agent, optionally one or more pharmaceutically acceptable carriers, and one or more TRPV1 antagonists or a physiologically acceptable salt thereof.

An additional aspect of the present invention is directed to a food product comprising one or more TRPV1 antagonists or a physiologically acceptable salt thereof.

An additional aspect of the present invention is directed to a method of increasing the palatability of food and its intake comprising administering one or more TRPV1 antagonists to a subject in need of such treatment.

These and additional aspects of the present invention are described in detail below.

### BRIEF DESCRIPTION OF THE FIGURES

The accompanying drawings, which are incorporated herein and form a part of the specification, serve to explain the principles of the invention and to enable a person skilled in the pertinent art to make and use the invention.

FIG. 1 illustrates the preferential activation of TRPV1 by selected, readily soluble drugs through a cell-based fluorescence assay.

FIG. 2 shows that the TPRV1 antagonist BCTC is capable of blocking cell membrane depolarization produced by either of the TRPV1 agonists, capsaicin or cetirizine. The experimental method requires the use of the Fluorescence Imaging Plate Reader (FLIPR) system to monitor fluorescence changes with respect to time.

FIG. 3 illustrates the specific inhibition of TRPV1 by a known TRPV1 antagonist in the presence of TRPV1 activators through a cell-based fluorescence assay. The TRPV1 antagonist, N-(4-tert-butylphenyl)-4-(3-chloropyridin-2-yl)tetrahydropryazine-1(2H)-carboxamide (BCTC), blocks TRPV1 activation.

FIG. 4 illustrates the effectiveness of a TRPV1 antagonist BCTC in reducing oral aversiveness to capsaicin in mice.

FIG. 5 illustrates the effects of the TRPV1 antagonists AMG9810 and SB366971 in reducing oral aversiveness to capsaicin in mice.

### DETAILED DESCRIPTION OF THE INVENTION

The present invention is generally directed to methods of inhibiting a taste by using a TRPV1 antagonist and compositions useful therefor.

In particular, certain aspects of the invention are directed to reducing the aversive taste associated with certain food, pharmaceutical, cosmetic, and other products. It has been found by the inventors of the present invention that the aversive tastes can be reduced, attenuated, or eliminated by administration of a TRPV1 antagonist. An unpleasant, or aversive, taste produced when certain products are consumed or used can cause nonoptimal use of the products. For example, certain pharmaceutical products contain an active pharmaceutical ingredient that causes an unpleasant taste when used. The unpleasant taste can cause noncompliance by the subject. The subject, often a patient, will choose not to use the pharmaceutical product because of the unpleasant taste. This is particularly problematic in children who often do not appreciate the beneficial effects of the pharmaceutical product.

In other aspects, as explained in more detail below, the invention is directed to the use of TRPV1 antagonists to reduce, attenuate, or eliminate an aversive taste, such as a bitter or hot/peppery taste, in a food product. In certain food products, it is undesirable to have an unpleasant, or aversive, taste, such as bitter, irritating or hot/peppery taste, associated with the food product when it is consumed. Other times, it is desirable to have a food product that has a hot/peppery, bitter, or sour taste for a limited amount of time, for example, an initial burst of hot/peppery, sour, or bitter flavor, followed by rapid diminution of that taste. Such a food product can be achieved using the methods and compositions of the invention as described in more detail herein.

### Methods of Use

A first aspect of the present invention is directed to a method of inhibiting an unpleasant taste, said method comprising administering to a subject in need of said inhibition an effective amount of a TRPV1 antagonist. The method in its various embodiments may be used to inhibit one or more tastes selected from the group consisting of bitter, sour, hot/peppery, irritating, pungent, and astringent. In a preferred embodiment, the method of the present invention inhibits a bitter and/or hot/peppery taste. In another embodiment, the method does not include inhibiting a salty taste.

The method of the invention can be practiced by administering one or more TRPV1 antagonists, as described herein. A “TRPV1 antagonist” is a substance which inhibits the function of the TRPV1 ion channel. The TRPV1 antagonist can eliminate or attenuate the activity of the TRPV1 ion channel. In certain embodiments, the TRPV1 antagonist inhibits the effect of an agonist which activates the vanilloid receptor, thereby inhibiting the function of TRPV1 as an ion channel. The TRPV1 antagonist of the present invention does not necessarily bind to the agonist-binding site on the vanilloid receptor but may still inhibit the function of the TRPV1 channel through non-competitive or uncompetitive mechanisms. Specifically, agonists which act on the vanilloid receptor include capsaicin, capsaicin derivatives, acid stimulation (proton), heat stimulation, and other small molecule compounds. The TRPV1 antagonist may be a substance which inhibits the Ca²⁺ inflow into the cell caused by the stimulation of TRPV1 by such agonists as capsaicin or other small molecule compounds, acid stimulation (proton) or heat stimulation. The TRPV1 antagonist may be a substance which prevents cell membrane depolarization caused by the stimulation of TRPV1 by such agonists as capsaicin or other small molecule compounds, acid stimulation (proton) or heat stimulation.

The vanilloid receptor subtype 1 (TRPV1) is the molecular target of capsaicin, an active ingredient in hot peppers. The molecular cloning of TRPV1 had been reported by Caterina, M. J., et al., Nature. 389(6653):816-24 (1997). TRPV1 is a non-selective cation channel which is activated or sensitized by different stimuli, including capsaicin, resiniferatoxin, heat, acid stimulation, products of lipid bilayer metabolism, anandamide, and lipoxygenase metabolites. See Prenikumar, et al., Nature. 408(6815):985-90 (2000); Szallasi, et al., Br. J. Pharmacol. 128(2):428-34 (1999); Gauldie et al, Br. J. Pharmacol. 2001 132(3):617-21 (2001); Olah, et al., J. Biol. Chem. 276(33):31163-70 (2001); and Hwang et al, Proc. Natl. Acad. Sci. U.S.A. 97(11):6155-60 (2000). TRPV1 is highly expressed in primary sensory neurons in rats, mice, and humans. See Caterina, M. J., et al., Nature. 389(6653):816-24 (1997); Xin, H., et al., Biochem. Biophys. Res. Commun. 332(3):756-62 (2005); Mezey, E., et al., Proc. Natl. Acad. Sci. U.S.A. 97(7):3655-60 (2000); Helliwell, R. J., et al, Neurosci. Lett. 250(3):177-80 (1998); Cortright, D. N., et al., Biochem. Biophys. Res. Commun. 281(5):1183-9. (2001). These sensory neurons innervate visceral organs including the dermis, bones, bladder, gastrointestinal tract, and lungs. TRPV1 is also expressed in other neuronal and non-neuronal tissues including but not limited to, CNS nuclei, kidney, stomach, and T-cells. See Nozawa, Y., et al., Neurosci. Lett. 309(1):33-6 (2001); Yiangou, Y., et al., B. J U. Int. 87(9):774-9 (2001); Birder, L. A., et al, Proc. Natl. Acad. Sci. U.S.A. 98(23):13396-401 (2001).

In accordance with the present invention, the method comprises using a TRPV1 antagonist to inhibit a taste, such as bitter, sour, hot/peppery, irritating, pungent, and astringent. Any TRPV1 antagonist which inhibits the activity of a TRPV1 receptor can be used in the present invention. Numerous TRPV1 antagonists are known to those of skill in the art. Specific examples of TRPV1 antagonists which can be used in the present invention are described in further detail herein. Furthermore, one of skill in the art is able to determine whether a given compound is a TRPV1 antagonist by using an appropriate assay as described in, for example, U.S. Pat. No. 6,780,650.

In certain embodiments, the TRPV1 antagonist may be a protein, a peptide, a small molecule, or a natural product. In a preferred instance, a small molecule TRPV1 antagonist is used to inhibit a taste in the method of the invention. For example, a TRPV1 antagonist may be a small molecule compound with a molecular weight of less than or equal to approximately 500 mass units. In another embodiment, a TRPV1 antagonist may be a small molecule with a molecular weight of about 50 to about 500 mass units. Alternatively, a TRPV1 antagonist may be a small molecule having a molecular weight of about 100, 200, 300, or 400 mass units. Such compounds can be selected from any of the specific compounds or groups described herein.

A TRPV1 antagonist useful in the present invention may include any number of chemical functional groups. In certain embodiments of the method, a preferred TRPV1 antagonist will include one or more functional groups selected from a preferred group. By way of example, in a preferred instance, a TRPV1 antagonist used in the method will contain about 5 or fewer hydrogen bond donors (e.g., OH and NH groups). In another embodiment, a TRPV1 antagonist used in the method will contain about 10 or fewer hydrogen bond acceptors (e.g., N and O). Such compounds can be selected from any of the specific compounds or groups described herein. In a preferred embodiment, the TRPV1 antagonist used in the present methods will contain 1 to 5 hydrogen bond donors and 1 to 5 hydrogen bond acceptors.

A TRPV1 antagonist may include, by way of nonlimiting examples, one or more of the following functional groups in its structure: pyridinyl, homopyridinyl, aminopiperidinylcarbamate, phenylcarbamate, cyclohexyl, cyclopentyl, piperidinyl, piperazinyl, pyrrolyl, furanyl thienyl, pyrazolyl, imidazolyl, thiazolyl, isothiazolyl, oxazolyl, isoxazolyl, oxadiazolyl, triazolyl, tetrazolyl, pyridyl, pyramidal, benzimidazolyl, naphthyl, indolyl, isoindolyl, benzofuranyl, isobenzofuranyl, benzo[b]thiophenyl, benz[d]isoxazoly, quinolinyl, isoquinolinyl, cinnolinyl, quinazolinyl, and quinoxalinyl groups. In another embodiment, a TRPV1 antagonist may include, but is not limited to, the following functional groups in its chemical structure: pyridinyl, homopyridinyl, aminopiperidinylcarbamate, phenylcarbamate, cyclohexyl, cyclopentyl, piperidinyl, piperazinyl, pyrrolyl, furanyl thienyl, pyrazolyl, imidazolyl, thiazolyl, isothiazolyl, oxazolyl, isoxazolyl, oxadiazolyl, triazolyl, tetrazolyl, pyridyl, pyramidal, benzimidazolyl, naphthyl, indolyl, isoindolyl, benzofuranyl, isobenzofuranyl, benzo[b]thiophenyl, benz[d]isoxazoly, quinolinyl, isoquinolinyl, cinnolinyl, quinazolinyl, and quinoxalinyl groups optionally substituted with benzene, halide, amine, hydroxyl and/or alkyl groups. Such compounds can be selected from any of the specific compounds or groups described herein.

In other embodiments, the TRPV1 antagonist used in the present method will contain a partition coefficient (log P) of about 0 to about 5, preferrably from about 1 to about 5, or from about 2 to about 4. Such compounds can be selected from any of the specific compounds or groups described herein.

In yet another embodiment, a suitable TRPV1 antagonist is a TRPV1 antagonist with a molecular weight of 100 to 500 mass units and which contains one or more, preferably one to three, of the following functional groups in its chemical structure: pyridinyl, homopyridinyl, aminopiperidinylcarbamate, phenylcarbamate, cyclohexyl, cyclopentyl, piperidinyl, piperazinyl, pyrrolyl, furanyl thienyl, pyrazolyl, imidazolyl, thiazolyl, isothiazolyl, oxazolyl, isoxazolyl, oxadiazolyl, triazolyl, tetrazolyl, pyridyl, pyramidal, benzimidazolyl, naphthyl, indolyl, isoindolyl, benzofuranyl, isobenzofuranyl, benzo[b]thiophenyl, benz[d]isoxazoly, quinolinyl, isoquinolinyl, cinnolinyl, quinazolinyl, and quinoxalinyl groups optionally substituted with benzene, halide, amine, hydroxyl and/or alkyl groups. Such compounds can be selected from any of the specific compounds or groups described herein.

In another embodiment, a preferred TRPV1 antagonist for use in the present invention is a compound which has low systemic bioavailability upon administration.

The present invention involves the use of TRPV1 antagonists for the local use of inhibiting one or more tastes. Accordingly, a TRPV1 antagonist that is effective locally (e.g., in the mouth) but has low systemic bioavailability is preferred. For example, a TRPV1 antagonist which has a systemic bioavailability of less than about 10%, 5%, 3%, or 1% is preferred, although TRPV1 antagonists having higher bioavailabilities can be used in the methods. Such compounds can be selected from any of the specific compounds or groups described herein.

Systemic bioavailability can be measured according to methods known to one of ordinary skill in the art. For example, the assay disclosed in U.S. Pat. No. 6,800,645 at col. 124, herein incorporated by reference in its entirety, can be used. Briefly, a suspension of the TRPV1 antagonist in 1.5% aqueous carboxymethylcellulose can be introduced into the stomach of a female Balb/c mouse by gavage at a dose of 1 mg/kg body weight. Following oral dosing, blood is obtained by cardiac puncture at a single time interval post-dose for each animal following carbon dioxide asphyxiation. Three animals are sacrificed at each time interval. Blood samples are obtained at the following times intervals after dosing; 0.25, 0.5, 1, 2, 3, 4, 5.5, 7 and 24 hours. Corresponding plasma is obtained by centrifugation of each blood sample. The TRPV1 antagonist content in the plasma samples is then determined using conventional methods.

Additionally, in certain embodiments of the invention, the method preferably uses a TRPV1 antagonist which is acid-labile. Such an antagonist can act locally (e.g., in the mouth), but, upon further transport to the acidic environment of the stomach, become inactive. By way of example, suitable acid-labile TPRV1 antagonists are those which have a half-life of less than about 1 hour in a pH of about 4 or less. Other antagonists with shorter half-lives in acid environments can likewise be used, for example, a TRPV1 antagonist having a half-life about 30 minutes or less in a pH of about 4 or less. Such compounds can be selected from any of the specific compounds or groups described herein.

In other instances of the present invention, the method of inhibiting a taste, such as bitter, sour, pungent, astringent, or hot/peppery, uses a TRPV1 antagonist with a Kᵢ of less than 1 micromolar. In another instance, the TRPV1 antagonist is a reversible antagonist with a rapid dissociation. Such compounds can be selected from any of the specific compounds or groups described herein.

In yet a further embodiment, the method of inhibiting a taste uses a TRPV1 antagonist which is (a) acid-labile, e.g., has a half-life of less than about 1 hour in a pH of about 4 or less; (b) has a K; of less than 1 micromolar; (c) has low systemic bioavailability, e.g., a systemic bioavailability of less than about 10%, 5%, 3%, or 1%. Such compounds can be selected from any of the specific compounds or groups described herein.

Numerous examples of TRPV1 antagonists are known. In one embodiment of the present invention, compounds which can be used in the method include compounds disclosed in U.S. Pat. No. 6,723,730, which is hereby incorporated by reference in its entirety. Specifically, such compounds include a compound having the formula:

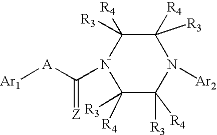

or a pharmaceutically acceptable salt thereof, wherein:
A is absent or is selected from the group consisting of O, S, NR_{A}, NR_{A}CR_{B}R_{B}′, C_{R}B_{R}B′NR_{A}, —CR_{A}═CR_{B}—, and C₃H₄; where R_{A}, R_{B}, and R_{B}′ are independently selected at each occurrence from hydrogen or alkyl;
Z is oxygen or sulfur;
each R₃ and R₄ independently are
   (a) selected from the group consisting of hydrogen; halogen; hydroxy; amino; cyano; nitro; —COOH; —CHO, optionally substituted alkyl; optionally substituted alkenyl; optionally substituted alkynyl; optionally substituted alkoxy; optionally substituted alkylthio; optionally substituted alkyl ketone; optionally substituted alkylester; optionally substituted alkylsulfinyl; optionally substituted alkylsulfonyl; optionally substituted mono- or di-alkylcarboxamide; optionally substituted —S(O)ₙNHalkyl; optionally substituted —S(O)ₙN(alkyl)(alkyl); optionally substituted —NHC(═O)alkyl; optionally substituted —NC(═O)(alkyl)(alkyl); optionally substituted —NHS(O), alkyl; optionally substituted —NS(O)ₙ(alkyl)(alkyl); optionally substituted saturated heterocyclic ring or partially unsaturated heterocycle of from 5 to 8 atoms, which saturated heterocyclic ring or partially unsaturated heterocycloalkyl contains 1, 2, or 3 heteroatoms selected from N, O, and S; optionally substituted aryl having from 1 to 3 rings; or optionally substituted heteroaryl, said heteroaryl having from 1 to 3 rings, 5 to 8 ring members in each ring and, in at least one of said rings, from 1 to about 3 heteroatoms per ring independently selected from the group consisting of N, O, and S; or
   (b) joined to a R₃ and R₄ not attached to the same carbon may be joined to form an optionally substituted aryl ring, a saturated or partially unsaturated carbocyclic ring of from 5 to 8 members, which carbocyclic ring is optionally substituted, or a saturated, partially unsaturated, or aromatic heterocyclic ring of from 5 to 8 members, which heterocyclic ring is optionally substituted and contains 1, 2, or 3 heteroatoms selected from N, O, and S;
Ar1 is selected from the group consisting of:
   (a) cyclohexyl, cyclopentyl, piperidinyl, piperazinyl, phenyl, pyrrolyl, furanyl, thienyl, pyrazolyl, imidazolyl, thiazolyl, isothiazolyl, oxazolyl, isoxazolyl, oxadiazolyl, triazolyl, tetrazolyl, pyridyl, pyramidal, pyrazinyl, benzimidazolyl naphthyl, indolyl, isoindolyl, benzofuranyl, isobenzofuranyl, benzo[b]thiophenyl, benz[d]isoxazoly, quinolinyl, isoquinolinyl, cinnolinyl, quinazolinyl, and quinoxalinyl, each of which is optionally mono-, di-, or trisubstituted with R₅ and
   (b) bicyclic oxygen-containing groups of the formula:

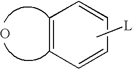

optionally mono-, di-, or trisubstituted with R₅, where L represents point of attachment and may be at any point on the benzene ring, and the oxygen-containing ring of the bicyclic oxygen-containing group consists of from 5 to 8 ring atoms, contains 1 or 2 oxygen atoms and remaining ring atoms are carbon;
Ar2 is selected from the group consisting of:
   (a) cyclohexyl, cyclopentyl, piperidinyl, piperazinyl, pyrrolyl, furanyl thienyl, pyrazolyl, imidazolyl, thiazolyl, isothiazolyl, oxazolyl, isoxazolyl, oxadiazolyl, triazolyl, tetrazolyl, pyridyl, pyramidal, benzimidazolyl, naphthyl, indolyl, isoindolyl, benzofuranyl, isobenzofuranyl, benzo[b]thiophenyl, benz[d]isoxazoly, quinolinyl, isoquinolinyl, cinnolinyl, quinazolinyl, and quinoxalinyl, each of which is optionally mono-, di-, or trisubstituted with R₅; and
   (b) bicyclic oxygen-containing groups of the formula:

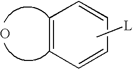

optionally mono-, di-, or trisubstituted with R₅, where L represents point of attachment and may be at any point on the benzene ring, and the oxygen-containing ring of the bicyclic oxygen-containing group consists of from 5 to 8 ring atoms, contains 1 or 2 oxygen atoms and remaining ring atoms are carbon;
R₅ is independently selected at each occurrence from the group consisting of halogen, nitro, halo(C₁₋₆)alkyl, halo(C₁₋₆)alkoxy, hydroxy, amino, C₁₋₆alkyl substituted with 0-2 R₆, C₂₋₆ alkenyl substituted with 0-2 R₆, C₂₋₆alkynyl substituted with 0-2 R₆, C₁₋₆ alkoxy and Y;
R₆ is independently selected at each occurrence from the group consisting of halogen, hydroxy, cyano C₁₋₄ alkyl, C₁₋₄ alkoxy, —S(O)ₙ(C₁₋₄ alkyl), halo(C₁₋₄)alkyl, halo(C₁₋₄)alkoxy, CO(C₁₋₄alkyl), CONH(C₁₋₄ alkyl), CON(C₁₋₄ alkyl₁) (C₁₋₄ alkyl₂) where alkyl₁ and alkyl₂ may be joined to form a saturated heterocyclic ring of from 5 to 8 ring atoms and containing 1, 2, or 3 heteroatoms independently selected from N, O, and S, —XR₇, and Y; X is independently selected at each occurrence from the group consisting of —CH₂—, —CHR₈—, —O—, —S(O)ₙ—, —NH—, —NR₈—, —C(═O)—, —C(═O)O—, —C(═O)NH—, —C(═O)NR₈—, —S(O)ₙNH—, —S(O)ₙ NR₈—, NHC(═O)—, —NR₈C(═O)—, —NHS(O)ₙ—, and —NR₈S(O)ₙ—;
R₇ and R₈ are independently selected at each occurrence from hydrogen, and straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms is unsubstituted or substituted with one or more substituent(s) independently selected from oxo, hydroxy, halogen, amino, cyano, nitro, haloalkyl, haloalkoxy, —O(alkyl), —NH(alkyl), —N(alkyl)(alkyl), —NHC(O)(alkyl), —N(alkyl)C(O)(alkyl), —NHS(O)ₙ(alkyl), —S(O)ₙ(alkyl), —S(O)ₙNH(alkyl), —S(O)ₙN(alkyl₃)(alkyl₄) where alkyl₃ and alkyl₄ are optionally joined to form a saturated heterocyclic ring consisting of from 5 to 8 ring atoms and containing 1, 2, or 3 heteroatoms independently selected from N, O, and S, and Y′; Y and Y′ are independently selected at each occurrence from 3- to 8-membered carbocyclic or heterocyclic groups which are saturated, unsaturated, or aromatic, which may be further substituted with one or more substituents independently selected from halogen, oxo, hydroxy, amino, nitro, cyano, alkyl, alkoxy, haloalkyl, haloalkoxy, mono- or dialkylamino, and alkylthio; wherein said 3- to 8-membered heterocyclic groups contain one or more heteroatom(s) independently selected from N, O, and S; n is independently chosen at each occurrence from 0, 1, and 2.

In other embodiments, the method of the present invention uses a compound selected from any of the specific compounds, groups, genera described in U.S. Pat. No. 6,723,730. Such groups, genera and specific compounds are described, for example, at cols. 3-18, 22-25, and 32-94 and in the claims of U.S. Pat. No. 6,723,730, said disclosures are likewise incorporated by reference herein.

In another embodiment, compounds which can be used in the method of inhibiting a taste, such as hot/peppery, bitter, or sour, include compounds disclosed in U.S. Pat. No. 7,015,233, which is hereby incorporated by reference in its entirety. Specifically, such compounds include a compound having the formula:

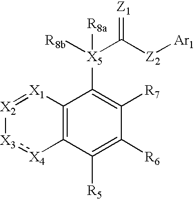

or a pharmaceutically acceptable salt or prodrug thereof, wherein:
is absent or a single bond;
X₁ is N or CR₁;
X₂ is N or CR₂;
X₃ is N, NR₃, or CR₃;
X₄ is a bond, N, or CR₄;
X₅ is N; provided that at least one of X₁, X₂, X₃, and X₄ is N;
Z₁ is O;
Z₂ is NH;
Ar₁ is dihydro-1H-indenyl, 1H-indenyl, tetrahydronaphthalenyl, or dihydronaphthalenyl, wherein the Ar₁ group is optionally substituted with 1, 2, 3, 4, or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, formyl, formylalkyl, haloalkoxy, haloalkyl, haloalkylthio, halogen, heterocycle, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl or (NZ_{A}Z_{B})sulfonyl, wherein Z_{A} and Z_{B} are each independently hydrogen, alkyl, alkylcarbonyl, formyl, aryl, or arylalkyl;
R₁, R₃, R₅, R₆, and R₇ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl or (NZ_{A}Z_{B})sulfonyl;
R₂ and R₄ are each independently hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})alkylcarbonyl, (NZ_{A}Z_{B})carbonyl, (NZ_{A}Z_{B})carbonylalkyl, (NZ_{A}Z_{B})sulfonyl, (NZ_{A}Z_{B})C(═NH)—, (NZ_{A}Z_{B})C(═NCN)NH—, or (NZ_{A}Z_{B})C(═NH)NH—; R_{A} is hydrogen or alkyl; R_{B} is alkyl, aryl, or arylalkyl;
R^{8a} is hydrogen or alkyl; and
R^{8b} is absent.

In other embodiments, the method of the present invention uses a compound selected from any of the specific compounds, groups, or genera described in U.S. Pat. No. 7,015,233. Such groups, genera, and specific compounds are described, for example, at cols. 2-16 and 24-35 and in the claims of U.S. Pat. No. 7,015,233, said disclosures are likewise incorporated by reference herein.

In another embodiment, compounds which can be used in the method of inhibiting a taste, such as hot/peppery, bitter, or sour, include compounds disclosed in U.S. Pat. No. 7,037,927, which is hereby incorporated by reference in its entirety. Specifically, such compounds include a compound having the formula:

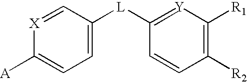

or a pharmaceutically acceptable salt, amide, ester, or prodrug thereof, wherein:
A is isoxazolyl, isothiazolyl, oxadiazolyl, oxazolyl, pyridinyl, pyridazinyl, pyrimidinyl, pyrazinyl, pyrazolyl, pyrrolyl, thiazolyl or thienyl,
X is CH; Y is CH;
L is —C(O)N(R₃)—;
R₁ is hydrogen, alkoxy, alkyl, aryloxy, haloalkoxy, haloalkyl, or halogen;
R₂ is hydrogen, alkoxy, alkyl, haloalkoxy, haloalkyl, or halogen; and
R₃ is hydrogen.

In other embodiments, the method of the present invention uses a compound selected from any of the specific compounds, groups, or genera described in U.S. Pat. No. 7,037,927. Such groups, genera, and specific compounds are described, for example, at cols. 2-10 and 17-32 and in the claims of U.S. Pat. No. 7,037,927, said disclosures are likewise incorporated by reference herein.

In another embodiment, compounds which can be used in the method of inhibiting a taste, such as hot/peppery, bitter, or sour, include compounds disclosed in U.S. Patent Publication No. 2004/0235853, which is hereby incorporated by reference in its entirety. Specifically, such compounds include a compound having the formula:

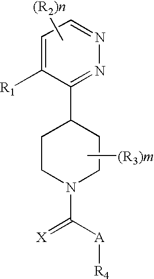

or a pharmaceutically acceptable salt thereof, wherein:
A is —NH—, —N(C₁-C₆)alkyl-, or —N—(O—C₁-C₆ alkyl)-;
R₁ is -halo, —CH₃, —NO₂, —CN, —OH, —OCH₃, —NH₂, C(halo)₃, —CH(halo)₂, or —CH₂(halo);
each R₂ is independently:
   (a) -halo, —OH, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₉-C₁₄)tricycloalkenyl,
   -(3- to 7-membered)heterocycle, or -(7- to 10-membered)bicycloheterocycle-,
   each of which is unsubstituted or substituted with one or more R₅ groups; or
   (c) -phenyl, -naphthyl, —(C₁₄)aryl or -(5- to 10-membered)heteroaryl, each of which is unsubstituted or substituted with one or more R₆ groups;
each R₃ is independently:
   (a) -halo, —CN, —OH, —NO₂, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle-,
   each of which is unsubstituted or substituted with one or more R₅ groups; or
   (c) -phenyl, -naphthyl, —(C₁₄)aryl or -(5- to 10-membered)heteroaryl, each of which is unsubstituted or substituted with one or more R₆ groups;
R₄ is:
   (a) -(C₁-C₁₀)alky-1, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle-,
   each of which is unsubstituted or substituted with one or more R₅ groups; or
   (b) -phenyl, -naphthyl, —(C₁₄)aryl or -(5- to 10-membered)heteroaryl, each of which is unsubstituted or substituted with one or more R₆ groups;
each R₅ is independently —CN, —OH, —(C₁-C₆)alkyl, —(C₂-C₆)alkenyl, -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each R₆ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, —CH₂(halo), —CN, —OH,
   -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each R₇ is independently —H, —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂(halo);
each halo is independently —F, —Cl, —Br, or —I;
n is an integer ranging from 0 to 2; and
m is an integer ranging from 0 to 2.

In other embodiments, the method of the present invention uses a compound selected from any of the specific compounds, groups, or genera described in U.S. Patent Publication No. 2004/0235853. Such groups, genera and specific compounds are described, for example, on pages 5-34 and 49-53 and in the claims of U.S. Patent Publication No. 2004/0235853, said disclosures are likewise incorporated by reference herein.

In another embodiment, compounds which can be used in the method of inhibiting a taste, such as hot/peppery, bitter, or sour, include compounds disclosed in U.S. Patent Publication No. 2005/0059671, which is hereby incorporated by reference in its entirety. Specifically, such compounds include a compound having the formula:

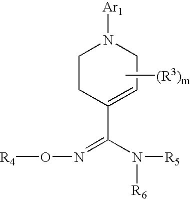

or a pharmaceutically acceptable salt thereof, wherein:
R₁ is -halo, —CH₃, —NO₂, —CN, —OH, —OCH₃, —NH₂, —C(halo)₃, —CH(halo)₂, or —CH₂(halo);
each R₂ is independently:
   (a) -halo, —CN, —OH, NO₂, —O(C₁-C₆)alkyl, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, -(C₈-C₁₄)tricycloalkyl, —(C₅-C₁₀)cycloalkenyl,
   -(C₈-C₁₄)bicycloalkenyl, —(C₉-C₁₄)tricycloalkenyl,
   -(3- to 7-membered)heterocycle, or -(7- to 10-membered)bicycloheterocycle, each of which is unsubstituted or substituted with one or more R₇ groups; or
   (c) -phenyl, -naphthyl, —(C₁₄)aryl or -(5- to 10-membered)heteroaryl, each of which is unsubstituted or substituted with one or more R₈ groups;
each R₃ is independently:
   (a) -halo, —CN, —OH, NO₂, —O(C₁-C₆)alkyl, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle-, each of which is unsubstituted or substituted with one or more R₇ groups; or
   (c) -phenyl, -naphthyl, —(C₁₄)aryl or -(5- to 10-membered)heteroaryl, each of which is unsubstituted or substituted with one or more R₈ groups;
R₄ is —H, —(C₁-C₁₀)alky-1, —C(O)R₉, or —C(O)NHR₉;
R₅ is —H or —(C₁-C₁₀)alkyl;
R₆ is:
   (a) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₅-C₁₄)bicycloalkenyl, —(C₈-C₁₄)tricycloalkenyl,
   -(3- to 7-membered)heterocycle, or -(7- to 10-membered)bicycloheterocycle-,
   each of which is unsubstituted or substituted with one or more R₇ groups; or
   (b) -phenyl, -naphthyl, —(C₁₄)aryl, or -(5- to 10-membered)heteroaryl, each of which is unsubstituted or substituted with one or more R₈ groups;
each R₇ and R₈ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, —CH₂(halo), —CN, —OH,
   -halo, —N₃, —NO₂, —N(R₁₀)₂, —CH═NR₁₀, —NR₁₀₀H, —OR₁₀, —COR₁₀, —C(O)OR₁₀,
   —OC(O)R₁₀, —OC(O)OR₁₀, —SR₁₀, —S(O)R₁₀, or —S(O)₂R₁₀; each R₉ is —H,
   —(C₁-C₆)alkyl, —(C₂-C₆)alkenyl, —(C₂-C₆)alkynyl, —(C₃-C₈)cycloalkyl,
   —(C₅-C₈)cycloalkenyl, -phenyl, -(3- to 5-membered)heterocycle, —C(halo)₃,
   —CH(halo)₂, —CH₂(halo), —OH, —N(R₁₀)₂, —CH═NR₁₀, —NR₁₀OH, —OR₁₀, or —SR₁₀;
each R₁₀ is independently —H, —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂(halo);
each halo is independently —F, —Cl, —Br, or —I;
n is an integer ranging from 0 to 2; and
m is an integer ranging from 0 to 2.

In other embodiments, the method of the present invention uses a compound selected from any of the specific compounds, groups, or genera described in U.S. Patent Publication No. 2005/0059671. Such groups, genera and specific compounds are described, for example, on pages 4-7, 10-22, 26-47, and 68-70 and in the claims of U.S. Patent Publication No. 2005/0059671, of which said disclosures are likewise incorporated by reference herein.

In another embodiment, compounds which can be used in the method of inhibiting a taste, such as hot/peppery, bitter, or sour, include compounds disclosed in U.S. Patent Publication No. 2005/0059671, which is hereby incorporated by reference in its entirety. Specifically, such compounds include a compound having the formula:

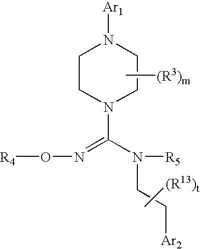

or a pharmaceutically acceptable salt thereof, wherein:
R₁ is -halo, —CH₃, —NO₂, —CN, —OH, —OCH₃, —NH₂, —C(halo)₃, —CH(halo)₂, or —CH₂(halo);
each R₂ is independently:
   (a) -halo, —CN, —OH, NO₂, —O(C₁-C₆)alkyl, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₅-C₁₄)tricycloalkenyl,
   -(3- to 7-membered)heterocycle, or -(7- to 10-membered)bicycloheterocycle, each of which is unsubstituted or substituted with one or more R₇ groups; or
   (c) -phenyl, -naphthyl, —(C₁₄)aryl or -(5- to 10-membered)heteroaryl, each of which is unsubstituted or substituted with one or more R₈ groups;
each R₃ is independently:
   (a) -halo, —CN, —OH, NO₂, —O(C₁-C₆)alkyl, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   -(C₈-C₁₄)bicycloalkenyl, —(C₈-C₁₄)tricycloalkenyl,
   -(3- to 7-membered)heterocycle, or
   -(7- to 10-membered)bicycloheterocycle-, each of which is unsubstituted or substituted with one or more R₇ groups; or
   (c) -phenyl, -naphthyl, -(C₁₄)aryl or -(5- to 10-membered)heteroaryl, each of which is unsubstituted or substituted with one or more R₈ groups;
R₄ is —H, -(C₁-C₁₀)alky-1, —C(O)R₉, or —C(O)NHR₉;
R₅ is —H or —(C₁-C₁₀)alkyl;
R₆ is:
   (a) -(C —C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle-,
   each of which is unsubstituted or substituted with one or more R₇ groups; or
   (b) -phenyl, -naphthyl, —(C₁₄)aryl, or -(5- to 10-membered)heteroaryl, each of
   which is unsubstituted or substituted with one or more R₈ groups;
      each R₇ and R₈ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, —CH₂(halo), —CN, —OH,
   -halo, —N₃, —NO₂, —N(R₁₀)₂, —CH═NR₁₀, —NR₁₀₀H, —OR₁₀, —COR₁₀, —C(O)OR₁₀,
   —OC(O)R₁₀, —OC(O)OR₁₀, —SR₁₀, —S(O)R₁₀, or —S(O)₂R₁₀;
each R₉ is —H, —(C₁-C₆)alkyl, —(C₂-C₆)alkenyl, —(C₂-C₆)alkynyl, —(C₃-C₈)cycloalkyl,
   —(C₅-C₈)cycloalkenyl, -phenyl, -(3- to 5-membered)heterocycle, —C(halo)₃,
   —CH(halo)₂, —CH₂(halo), —OH, —N(R₁₀)₂, —CH═NR₁₀, —NR₁₀₀H, —OR₁₀, or —SR₁₀;
each R₁₀ is independently —H, —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂(halo);
each halo is independently —F, —Cl, —Br, or —I;
p is an integer ranging from 0 to 2; and
m is an integer ranging from 0 to 2.

In other embodiments, the method of the present invention uses a compound selected from any of the specific compounds, groups, or genera described in U.S. Patent Publication No. 2005/0059671. Such groups, genera and specific compounds are described, for example, on pages 8-9, 22-24 and 26-47 and in the claims of U.S. Patent Publication No. 2005/0059671, said disclosures of which are likewise incorporated by reference herein.

In another embodiment, compounds which can be used in the method of inhibiting a taste, such as hot/peppery, bitter, or sour, include compounds disclosed in U.S. Patent Publication No. 2005/0059671, which is hereby incorporated by reference in its entirety. Specifically, such compounds include a compound having the formula:

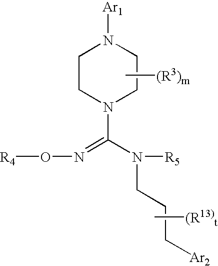

or a pharmaceutically acceptable salt thereof, wherein:
R₁ is -halo, —CH₃, —NO₂, —CN, —OH, —OCH₃, —NH₂, —C(halo)₃, —CH(halo)₂, or —CH₂(halo);
each R₂ is independently:
   (a) -halo, —CN, —OH, NO₂, —O(C₁-C₆)alkyl, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₉-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle, each of which is unsubstituted or substituted with one or more R₇ groups; or
   (c) -phenyl, -naphthyl, —(C₁₄)aryl or -(5- to 10-membered)heteroaryl, each of which is unsubstituted or substituted with one or more R₈ groups;
each R₃ is independently:
   (a) -halo, —CN, —OH, NO₂, —O(C₁-C₆)alkyl, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle-,
   each of which is unsubstituted or substituted with one or more R₇ groups; or
   (c) -phenyl, -naphthyl, —(C₁₄)aryl or -(5- to 10-membered)heteroaryl, each of which is unsubstituted or substituted with one or more R₈ groups;
R₄ is —H, -(C₁-C₁₀)alkynyl, —C(O)R₉, or —C(O)NHR₉;
R₅ is —H or —(C₁-C₁₀)alkyl;
R₆ is: (a) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₉-C₁₄)tricycloalkenyl,
   -(3- to 7-membered)heterocycle, or -(7- to 10-membered)bicycloheterocycle-,
   each of which is unsubstituted or substituted with one or more R₇ groups; or
   (b) -phenyl, -naphthyl, —(C₁₄)aryl, or -(5- to 10-membered)heteroaryl, each of which is unsubstituted or substituted with one or more R₈ groups;
each R₇ and R₈ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, —CH₂(halo), —CN, —OH,
   -halo, —N₃, —NO₂, —N(R₁₀)₂, —CH═NR₁₀, —NR₁₀OH, —OR₁₀, —COR₁₀, —C(O)OR₁₀, —OC(O)R₁₀, —OC(O)OR₁₀, —SR₁₀, —S(O)R₁₀, or —S(O)₂R₁₀;
each R₉ is —H, —(C₁-C₆)alkyl, —(C₂-C₆)alkenyl, —(C₂-C₆)alkynyl, —(C₃-C₈)cycloalkyl,
—(C₅-C₈)cycloalkenyl, -phenyl, -(3- to 5-membered)heterocycle, —C(halo)₃,
—CH(halo)₂, —CH₂(halo), —OH, —N(R₁₀)₂, —CH═NR₁₀, —NR₁₀H —OR₁₀, or —SR₁₀;
each R₁₀ is independently —H, —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂(halo);
each halo is independently —F, —Cl, —Br, or —I;
p is an integer ranging from 0 to 2; and
m is an integer ranging from 0 to 2.

In other embodiments, the method of the present invention uses a compound selected from any of the specific compounds, groups, or genera described in U.S. Patent Publication No. 2005/0059671. Such groups, genera and specific compounds are described, for example, on pages 9-10, 24-26 and 26-47 and in the claims of U.S. Patent Publication No. 2005/0059671, said disclosures of which are likewise incorporated by reference herein.

In another embodiment, compounds which can be used in the method of inhibiting a taste, such as hot/peppery, bitter, or sour, include compounds disclosed in PCT Publication WO 2005/012287, which is hereby incorporated by reference in its entirety. Specifically, such compounds include a compound having the formula:

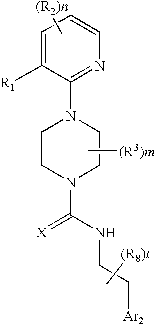

or a pharmaceutically acceptable salt thereof, wherein
X is S or O;
Ar2 is

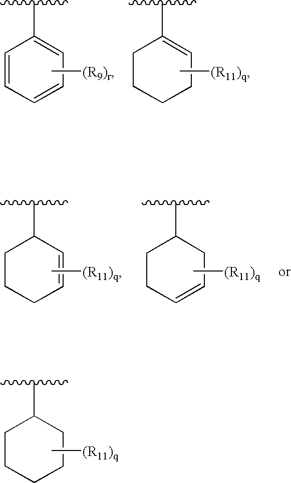

R₁ is —CF₃, —NO₂, or —CN;
each R₂ is independently:
   (a) -halo, —CN, —OH, —NO₂, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered) heterocycle, or -(7- to 10-membered)bicycloheterocycle, each of which is unsubstituted or substituted with one or more R₅ groups; or
   (c) -phenyl, -naphthyl, -(C₁₄)aryl or -(5- to 10-membered)heteroaryl, each of which is unsubstituted or substituted with one or more R₆ groups;
each R₃ is independently:
   (a) -halo, —CN, —OH, —NO₂, or —NH₂;
   (b) -(C₁-C₁₀)alkyl, —(C₂-C₁₀)alkenyl, —(C₂-C₁₀)alkenyl, —(C₃-C₁₀)cycloalkyl,
   —(C₈-C₁₄)bicycloalkyl, —(C₈-C₁₄)tricycloalkyl, —(C₅-C₁₀)cycloakenyl,
   —(C₈-C₁₄)bicycloalkenyl, —(C₈-C₁₄)tricycloalkenyl,
   -(3- to 7-membered) heterocycle, or -(7- to 10-membered) bicycloheterocycle,
each of which is unsubstituted or substituted with one or more R₅ groups; or
   (c) -phenyl,-naphthyl, —(C)₄)aryl or -(5- to 10-membered) heteroaryl, each of which is unsubstituted or substituted with one or more R₆ groups;
each R₅ is independently —CN, —OH, —(C₁-C₆) alkyl, —(C₂-C₆) alkenyl, —(C₂-C₆)alkynyl,
   -halo, —N₃, —NO₂, —N(R₇)₂, —CH═NR₇, —NR₇OH, —OR₇, —COR₇, —C(O)OR₇,
   —OC(O)R₇, —OC(O)OR₇, —SR₇, —S(O)R₇, or —S(O)₂R₇;
each R₆ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered) heterocycle, —C(halo)₃, —CH(halo)₂, —CH₂(halo), —CN, —OH,
   -halo, —N₃, —NO₂, —CH═NR₇, —NR₇OH, —OR₇, —COR₇, —C(O)OR₇, —OC(O)R₇,
   —OC(O)OR₇, —SR₇, —S(O)R₇, or —S(O)₂R₇;
each R₇ is independently —H, —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂ (halo);
each R⁸ is independently —(C₁-C₆)alkyl, —(C₂-C₆)alkenyl, —(C₂-C₆) alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂(halo);
each R⁹ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl, —C(halo)₃, —CH(halo)₂, or
   CH₂(halo), —CN, —OH, -halo, —N₃, —NO₂, —CH═NR₇, —NR₇OH, —OR₇, —COR₇,
   —C(O)OR₇, —OC(O)R₇, —OC(O)OR₇, —SR₇, —S(O)R₇, or —S(O)₂R₇;
each R₁₁ is independently —CN, —OH, —(C₁-C₆)alkyl, —(C₂-C₆)alkenyl, —(C₂-C₆)alkynyl, -halo, —N₃, —NO₂, —N(R₇)₂, —CH═NR₇, —NR₇OH, —OR₇, —COR₇, —C(O)OR₇,
   —OC(O)R₇, —OC(O)OR₇, —SR₇, —S(O)R₇, or —S(O)₂R₇;
each halo is independently —F, —Cl, —Br, or —I;
n is an integer ranging from 0 to 3;
m is 0 or 1;
q is an integer ranging from 0 to 6;
r is an integer ranging from 0 to 5; and
t is an integer ranging from 0 to 2.

In other embodiments, the method of the present invention uses a compound selected from any of the specific compounds, groups, or genera described in PCT Publication WO 2005/012287. Such groups, genera and specific compounds are described, for example, on pages 10-14 and 16-51 and in the claims of PCT Publication WO 2005/012287, of which portions are likewise incorporated by reference herein.

In another embodiment, compounds which can be used in the method of inhibiting a taste, such as hot/peppery, bitter, or sour, include compounds disclosed in PCT Publication WO 2005/012287, which is hereby incorporated by reference in its entirety. Specifically, such compounds include a compound having the formula:

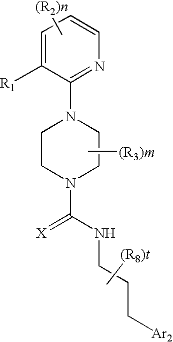

or a pharmaceutically acceptable salt thereof, wherein
X is S or O;
Ar2 is

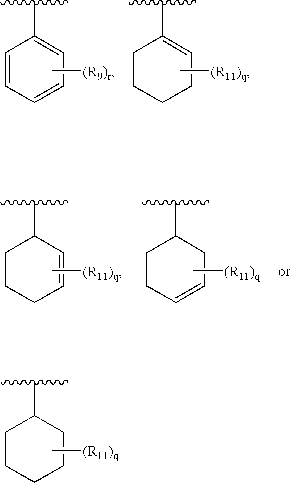

R₁ is —CF₃, —NO₂, or —CN;
each R₂ is independently:
   (a) -halo, —CN, —OH, —NO₂, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered) heterocycle, or -(7- to 10-membered)bicycloheterocycle, each of which is unsubstituted or substituted with one or more R₅ groups; or
   (c) -phenyl, -naphthyl, —(C₁₄)aryl or -(5- to 10-membered)heteroaryl, each of which is unsubstituted or substituted with one or more R₆ groups;
each R₃ is independently:
   (a) -halo, —CN, —OH, —NO₂, or —NH₂;
   (b) -(C₁-C₁₀)alkyl, —(C₂-C₁₀)alkenyl, —(C₂-C₁₀)alkenyl, —(C₃-C₁₀)cycloalkyl,
   -(C₈-C₁₄)bicycloalkyl, —(C₈-C₁₄)tricycloalkyl, —(C₅-C₁₀)cycloakenyl,
   —(C₈-C₁₄)bicycloalkenyl, —(C₈-C₁₄)tricycloalkenyl,
   -(3- to 7-membered) heterocycle, or -(7- to 10-membered) bicycloheterocycle, each of which is unsubstituted or substituted with one or more R₅ groups; or
   (c) -phenyl,-naphthyl, —(C₁₄)aryl or -(5- to 10-membered) heteroaryl, each of which is unsubstituted or substituted with one or more R₆ groups;
each R₅ is independently —CN, —OH, —(C₁-C₆) alkyl, —(C₂-C₆) alkenyl, —(C₂-C₆)alkynyl,
   -halo, —N₃, —NO₂, —N(R₇)₂, —CH═NR₇, —NR₇OH, —OR₇, —COR₇, —C(O)OR₇,
   —OC(O)R₇, —OC(O)OR₇, —SR₇, —S(O)R₇, or —S(O)₂R₇;
each R₆ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl, -(3- to 5-membered) heterocycle, —C(halo)₃, —CH(halo)₂, —CH₂(halo), —CN, —OH, -halo, —N₃, —NO₂, —CH═NR₇, —NR₇OH, —OR₇, —COR₇, —C(O)OR₇, —OC(O)R₇,
   —OC(O)OR₇, —SR₇, —S(O)R₇, or —S(O)₂R₇;
each R₇ is independently —H, —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl,
   -(3- to 5-membered)heterocycle, —C(halo)₃, —CH(halo)₂, or —CH₂ (halo);
each R₈ is independently —(C₁-C₆)alkyl, —(C₂-C₆)alkenyl, —(C₂-C₆) alkynyl,
—(C₃-C₈)cycloalkyl, —(C₅-C₈)cycloalkenyl, -phenyl,
   -(3- to 5-membered)heterocycle, —C(halo)₃, —CH(halo)₂, or —CH₂(halo);
each R₉ is independently —(C₁-C₆)alkyl, —(C₂-C₆)alkenyl, —(C₂-C₆)alkynyl,
   —(C₃-C₈)cycloalkyl, —(C₅-C₈)cycloalkenyl, -phenyl, —C(halo)₃, —CH(halo)₂, or CH₂(halo), —CN, —OH, -halo, —N₃, —NO₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each R₁, is independently —CN, —OH, —(C₁-C₆)alkyl, —(C₂-C₆)alkenyl, —(C₂-C₆)alkynyl, -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each halo is independently —F, —Cl, —Br, or —I;
n is an integer ranging from 0 to 3;
m is 0 or 1;
q is an integer ranging from 0 to 6;
r is an integer ranging from 0 to 5; and
t is an integer ranging from 0 to 2.

In other embodiments, the method of the present invention uses a compound selected from any of the specific compounds, groups, or genera described in PCT Publication WO 2005/012287. Such groups, genera, and specific compounds are described, for example, on pages 10-14 and 16-51 and in the claims of PCT Publication WO 2005/012287, said portions of which are likewise incorporated by reference herein.

In another embodiment, compounds which can be used in the method of inhibiting a taste, such as hot/peppery, bitter, or sour, include compounds disclosed in PCT Publication WO 2005/004866, which is hereby incorporated by reference in its entirety. Specifically, such compounds include a compound having the formula:

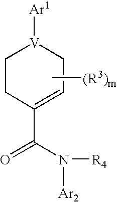

or a pharmaceutically acceptable salt thereof, wherein

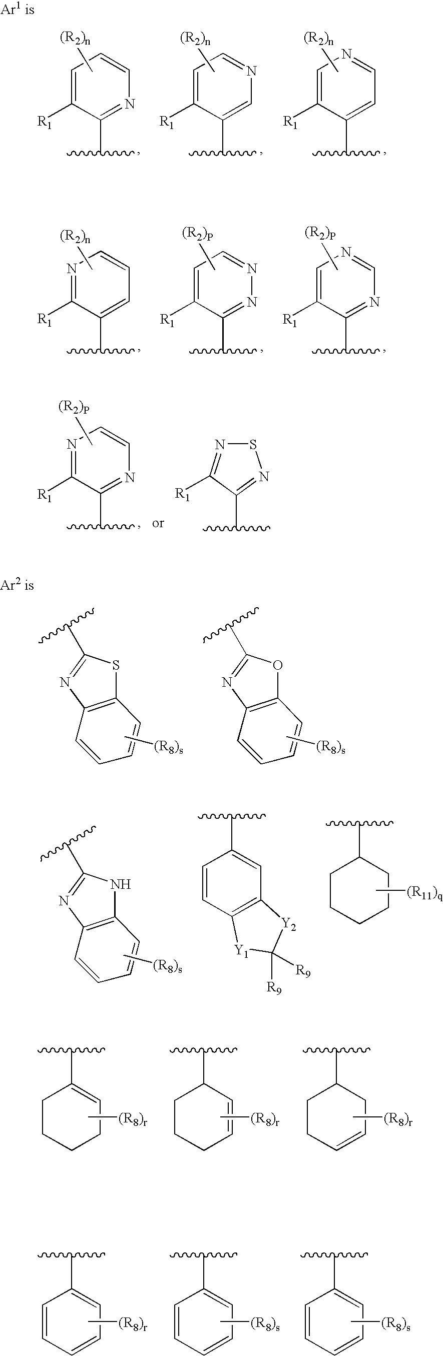

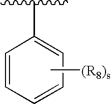

V is N or CH;
X is O or S;
Y₁ and Y₂ are —CH₂— and —CH₂—, —O— and —O—, —NH— and —NH—, —S— and —S—, —CH₂— and —O—, —CH₂— and —NH—, —CH₂— and —S—, —O— and —CH₂—, —NH— and —CH₂—, —S— and —CH₂—, —O— and —NH—, —NH— and —O—, —S— and —NH—, or —NH— and —S— respectively;
R₁ is —H, -halo, —(C₁-C₄)alkyl, —NO₂, —CN, —OH, —OCH₃, —NH₂, —C(halo)₃, —CH(halo)₂, or —CH₂(halo);
each R₂ is independently:
   (a) -halo, —CN, —OH, —NO₂, or —NH₂,
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle, each of which is unsubstituted or substituted with one or more R₅ groups, or
   (c) -phenyl, -naphthyl, —(C₁₄)aryl or -(5- to 10-membered)heteroaryl, each of which is unsubstituted or substituted with one or more R₆ groups;
each R₃ is independently:
   (a) -halo, —CN, —OH, —NO₂, or —NH₂,
   (b) -(C₁-C₁₀)alkyl, —(C₂-C₁₀)alkenyl, —(C₂-C₁₀)alkynyl, —(C₃-C₁₀)cycloalkyl,
   —(C₈-C₁₄)bicycloalkenyl, —(C₈-C₁₄)tricycloalkenyl,
   -(3- to 7-membered)heterocycle, or -(7- to 10-membered)bicycloheterocycle, each of which is unsubstituted or substituted with one or more R₅ groups, or
   (c) phenyl, -naphthyl, —(C₁₄)aryl or -(5- to 10-membered)heteroaryl, each of which is unsubstituted or substituted with one or more R₆ groups;
R₄ is —H, —(C₁-C₆)alkyl;
each R₅ is independently —CN, —OH, —(C₁-C₆)alkyl, —(C₂-C₆)alkenyl, —(C₂-C₆)alkynyl, -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, or S(O)₂R₇;
each R₆ is independently —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, -(3- to 5-membered)heterocycle, —C(halo)₃, —CH(halo)₃, —CH(halo)₂, or CH₂(halo);
each R₈ is independently —(C₁-C₁₀)alkyl, —(C₂-C₆)alkenyl, —(C₂-C₆)alkynyl, —(C₃-C₈)cycloalkyl, —(C₅-C₈)cycloalkenyl, -phenyl, —C(halo)₃, —CH(halo)₂, —CH₂(halo), —CN, —OH, -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, —S(O)₂R₇, —R₇OR₇, —R₇COR₇, —R₇C(O)OR₇, —R₇OC(O)R₇, —R₇OC(O)OR₇, —R₇SR₇, —R₇S(O)R₇, —R₇S(O)₂R₇, —C(halo)₂C(halo)₃, —C(halo)₂CH(halo)₂, —CH(C(halo)₃)₂, —CH(C(halo)₃)(CH₃), —OC(halo)₂C(halo)₃, OC(halo)₂CH(halo)₂, OCH(C(halo)₃)₂, —OCH(C(halo)₃)(CH₃), —C(OH)(CF₃)₂, -(C₁-C₁₀)alkyl, or -(3- to 7-membered)heterocycle;
each R₉ is independently —H, -halo or —(C₁-C₆)alkyl;
each R₁, is independently —CN, —OH, —(C₁-C₆)alkyl, —(C₂-C₆)alkenyl, —(C₂-C₆)alkynyl, -halo, —N₃, —NO₂, —N(R₇)₂, —CH═NR₇, —NR₇OH, —OR₇, —COR₇, —C(O)OR₇, —OC(O)R₇, or —OC(O)OR₇;
each halo is independently —F, —Cl, —Br, or —I;
m is 0 or 1 and when m is 1, R₃ is attached to the 2-, 3-, 5-, or 6-position of the cyclo(hetero)alkenyl ring;
n is an integer ranging from 0 to 3;
p is an integer ranging from 0 to 2;
q is an integer ranging from 0 to 6;
r is an integer ranging from 0 to 5; and
s is an integer ranging from 0 to 4.

In other embodiments, the method of the present invention uses a compound selected from any of the specific compounds, groups, or genera described in PCT Publication WO 2005/004866. Such groups, genera and specific compounds are described, for example, on pages 10-790, 836-845 and 864-874 and in the claims of PCT Publication WO 2005/004866, said portions of which are likewise incorporated by reference herein.

In another embodiment, compounds which can be used in the method of inhibiting a taste, such as hot/peppery, bitter, or sour, include compounds disclosed in U.S. Patent Publication No. 2004/0186111, which is hereby incorporated by reference in its entirety. Specifically, such compounds include a compound having the formula:

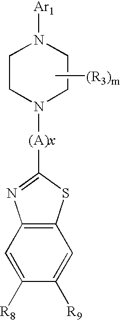

or a pharmaceutically acceptable salt thereof, wherein
Ar₁ is

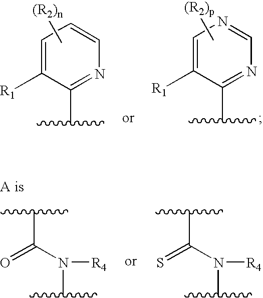

R₁ is —Cl, —Br, —I, —(C₁-C₆)alkyl, —NO₂, —CN, —OH, —OCH₃, —NH₂, —C(halo)₃, —CH(halo)₂, or —CH₂(halo);
each R₂ is independently:
   (a) -halo, —CN, —OH, —O(C₁-C₆)alkyl, —NO₂, or —NH₂;
   (b) -(C₁-C₁₀)alkyl, —(C₂-C₁₀)alkenyl, —(C₂-C₁₀)alkynyl, —(C₃-C₁₀)cycloalkyl,
   -(C₈-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle, each of which is unsubstituted or substituted with one or more R₅ groups; or
   (c) -phenyl, -naphthyl, -(C₁₄)aryl, or -(5- to 10-membered)heteroaryl, each of which is unsubstituted or substituted with one or more R₆ groups;
each R₃ is independently:
   (a) -halo, —CN, —OH, —O(C₁-C₆)alkyl, —NO₂, or —NH₂;
   (b) -(C₁-C₁₀)alkyl, —(C₂-C₁₀)alkenyl, —(C₂-C₁₀)alkynyl, —(C₃-C₁₀)cycloalkyl,
   —(C₉-C₁₄)bicycloalkyl, —(C₈-C₁₄)tricycloalkyl, —(C₅-C₁₀)cycloalkenyl,
   —(C₈-C₁₄)bicycloalkenyl, —(C₈-C₁₄)tricycloalkenyl,
   -(3- to 7-membered)heterocycle, or -(7- to 10-membered)bicycloheterocycle, each of which is unsubstituted or substituted with one or more R₅ groups; or
   (c) -phenyl, -naphthyl, —(C₁₄)aryl or -(5- to 10-membered)heteroaryl, each of which is unsubstituted or substituted with one or more R₆ groups;
R₄ is —H or —(C₁-C₆)alkyl;
each R₅ is independently —CN, —OH, -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each R₆ is independently —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH(halo)₂, —CH₂(halo), —CN, —OH, -halo, —N₃, —NO₂, —N(R₇)₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each R₇ is independently —H, —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH₂(halo), or —CH(halo)₂;
R⁸ and R₉ are each independently —H, —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —C(halo)₃, —CH(halo)₂, —CH₂(halo), —CN, —OH, -halo, —N₃, —N(R₇)₂, —CH═NR₇, —NR₇OH, —OR₇, —COR₇, —C(O)OR₇, —OC(O)R₇, —OC(O)OR₇, —SR₇, —S(O)R₇, or —S(O)₂R₇;
each -halo is —F, —Cl, —Br,- or —I;
n is an integer ranging from 0 to 3;
p is an integer ranging from 0 to 2;
m is 0 or 1; and
x is 0 or 1.

In other embodiments, the method of the present invention uses a compound selected from any of the specific compounds, groups, or genera described in U.S. Patent Publication No. 2004/0186111. Such groups, genera and specific compounds are described, for example, on pages 5-199, 227 and 232 and in the claims of U.S. Patent Publication No. 2004/0186111, said portions of which are likewise incorporated by reference herein.

In another embodiment, compounds which can be used in the method of inhibiting a taste, such as hot/peppery, bitter, or sour, include compounds disclosed in PCT Publication WO 2006/038070, which is hereby incorporated by reference in its entirety. Specifically, such compounds include a compound having the formula:

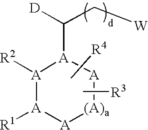

or a pharmaceutically acceptable salt thereof, wherein
A is, independently, either an sp²- or sp³-hybridized carbon or nitrogen atom;
D is selected from the group consisting of —H, —OH, halogen, —(CH₂)₀₋₆Y, —O—(CH₂)₀₋₆Y,
   wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹,
wherein R⁹ is selected from the group consisting of —H, —C₁₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹X¹, —SO₂NX¹X², wherein X¹ and X² are each, independently, H, aryl, or C₁-C₆— alkyl; salts thereof, esters thereof, and any combination thereof;
W is selected from the group consisting of —H, —OH, halogen, —(CH₂)₀₋₆Y, —O—(CH₂)₀₋₆Y, wherein Y is selected from —H, —CN —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹,
wherein R⁹ is selected from the group consisting of —H, —C₁₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; N(X¹X², —SO₂N(X¹)X², or —(CH₂)₁₋₆SO₂N(X¹) X², wherein X¹ and X² are each, independently, H, aryl, C₁-C₆-alkyl; salts thereof, esters thereof, and any combination thereof, and any combination thereof; or the formula

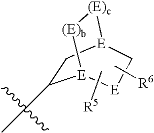

wherein E is, independently, either an sp²- or sp³-hybridized carbon or nitrogen atom;
R⁵ and R⁶ are each, independently, —H, —OH, —(CH₂)₀₋₆Y, —O—(CH₂)₀₋₆Y, wherein Y is selected from —CHCH₂, —CH₂CHCH₂, —H, —OH, —CN, halo, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹,
wherein R⁹ is selected from the group consisting of —H, —C₁-C₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹X², —SO₂N(X¹)X² wherein X¹ and X are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
   -GR¹⁰—CH₂—CH₂—
   —CH₂-GR¹⁰—CH₂—
—CH₂—CH₂-GR¹⁰-
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰-
-GR¹⁰═CH—CH═CH—
—CH=GR¹⁰—CH═CH-1
—CH═CH-GR¹⁰═CH—
—CH═CH—CH=GR¹⁰-
wherein G is either an sp²- or Sp³-hybridized carbon or nitrogen atom; wherein the meaning set forth for R⁶;
a, b, c and dare each 0 or 1.

In other embodiments, the method of the present invention uses a compound selected from any of the specific compounds, groups, or genera described in PCT Publication WO 2006/038070. Such groups, genera, and specific compounds are described, for example, on pages 5-12 and 22-34 and in the claims of PCT Publication WO 2006/038070, said portions of which are likewise incorporated by reference herein.

In another embodiment, compounds which can be used in the method of inhibiting a taste, such as hot/peppery, bitter, or sour, include compounds disclosed in U.S. Patent Publication No. 2006/0111337, which is hereby incorporated by reference in its entirety. Specifically, such compounds include a compound having the formula:

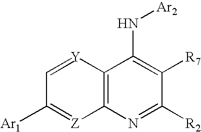

or a pharmaceutically acceptable salt thereof, wherein:
Y and Z are independently N or CR₁;
each R is independently hydrogen, halogen, cyano, amino, C₁-C₄alkyl, C₁-C₄haloalkyl, C₁-C₄alkoxy, C₁-C₄haloalkoxy or mono- or di-(C₁-C₄alkyl)amino;
R₂ is:
   (i) hydrogen, halogen or cyano;
   (ii) a group of the formula —R_{c}-M-R_{d}—R_{y}, wherein: & is C₀-C₃alkylene or is joined to R_{y} or R_{z}, to form a 4- to 10-membered carbocycle or heterocycle that is substituted with from 0 to 2 substituents independently chosen from R_{b}; M is absent, a single covalent bond, O, S, SO, SO₂, C(═O), OC(═O), C(═O)O, O—C(═O)O, C(═O)N(R_{z}), OC(═O)N(R_{z}), N(R_{z})C(═O), N(R_{z})C(═O)O, N(R_{z})SO₂, SO₂N(R_{z}) or N(R_{z}), such that M is not N(R_{z})C(═O)O if R_{c} is a single covalent bond; R_{d} is absent, a single covalent bond or C₁-C₈alkylene substituted with from 0 to 3 substituents independently chosen from R_{b}; and R_{y} and R_{z} if present, are: (a) independently hydrogen, C₁-C₈alkyl, C₂-C₈alkyl ether, C₂-C₈alkenyl, a 4- to 10-membered carbocycle or heterocycle, or joined to R_{c}C to form a 4- to 10-membered carbocycle or heterocycle, wherein each non-hydrogen R_{y} and R₁ is substituted with from 0 to 6 substituents independently chosen from R_{b}; or
   (b) joined to form a 4- to 10-membered carbocycle or heterocycle that is substituted with from 0 to 6 substituents independently chosen from R_{b}; such that if Y and Z are both N, then R₂ is not NH₂; or
   (iii) taken together with R₇ to form a fused 5- to 7-membered ring that is substituted with from 0 to 3 substituents independently chosen from oxo and C₁-C₄alkyl;
R₇ is hydrogen, halogen, COOH, C₁-C₄alkyl, C₁-C₄haloalkyl, C₁-C₄alkoxy, C₁-C₄alkoxycarbonyl or taken together with R₂ to form a fused, optionally substituted ring;
Ar₁ is phenyl or 6-membered heteroaryl, each of which is: (i) substituted at one ring carbon atom meta or para to the point of attachment with one substituent chosen from halogen, cyano, nitro and groups of the formula LRₐ, and
   (ii) optionally substituted at any other ring carbon atom(s) with an additional 1-3 substituents independently chosen from halogen, cyano, nitro and groups of the formula LRₐ;
Ar2 is 6- to 10-membered aryl or 5- to 10-membered heteroaryl, each of which is substituted with from 0 to 6 substituents independently chosen from oxo and groups of the formula LRₐ;
L is independently selected at each occurrence from a single covalent bond, O, C(═O), OC(═O), C(═O)O, OC(═O)O, S(O)ₘ, N(Rₓ), C(═O)N(Rₓ), N(Rₓ)C(═O), N(Rₓ)S(O)ₘ, S(O)ₘN(Rₓ) and N[S(O)ₘR_{w}]S(O)ₘ; wherein m is independently selected at each occurrence from 0, 1 and 2; Rₓ is independently selected at each occurrence from hydrogen, C₁-C₆alkyl, C₁-C₆alkanoyl and C₁-C₆alkylsulfonyl; and R_{w} is C₁-C₆alkyl; Rₐ is independently selected at each occurrence from:
   (i) hydrogen, such that Rₐ is not hydrogen if L is a bond; and
   (ii) C₁-C₈alkyl, C₂-C₈alkenyl, C₂-C₈alkynyl, (C₃-C₈cycloalkyl)C₀-C₆alkyl, C₁-C₈haloalkyl, C₂-C₈alkyl ether, mono- and di-(C₁-C₈alkyl)amino and (3- to 10-membered heterocycle)C₀-C₆alkyl, each of which is substituted with from 0 to 6 substituents independently selected from R_{b}; and R_{b} is independently chosen at each occurrence from hydroxy, halogen, amino, aminocarbonyl, aminosulfonyl, cyano, nitro, oxo, COOH, C₁-C₈alkyl, C₁-C₈alkenyl, C₁-C₈alkynyl, C₁-C₈alkoxy, C₁-C₈alkylthio, C₁-C₈alkanoyl, C₂-C₈alkanoyloxy, C₁-C₈alkoxycarbonyl, C₁-C₈alkyl ether, C₁-C₈hydroxyalkyl, C₁-C₈haloalkyl, mono- or di-(C₁-C₆alkyl)aminoC₀-C₄alkyl, C₁-C₈alkylsulfonyl, mono- or di-(C₁-C₆alkyl)aminocarbonyl, mono- or di-(C₁-C₆alkyl)aminosulfonyl, (3- to 7-membered carbocycle)C₀-C₈alkyl and (4- to 7-membered heterocycle)C₀-C₈alkyl.

In other embodiments, the method of the present invention uses a compound selected from any of the specific compounds, groups, or genera described in U.S. Patent Publication No. 2006/0111337. Such groups, genera and specific compounds are described, for example, on pages 1-2, 8-12 and 29-132 and in the claims of U.S. Patent Publication No. 2006/0111337, said portions of which are likewise incorporated by reference herein.

In another embodiment, compounds which can be used in the method of inhibiting a taste, such as hot/peppery, bitter, or sour, include compounds disclosed in U.S. Patent Publication No. 2005/0165049, which is hereby incorporated by reference in its entirety. Specifically, such compounds include a compound having the formula:

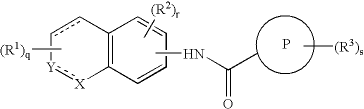

or a pharmaceutically acceptable salt or solvate thereof, wherein:
P is selected from phenyl, heteroaryl or heterocyclyl;
R¹ and R² are independently selected from halo, alkyl, alkoxy, cycloalkyl, aralkyl, aralkoxy, cycloalkylalkyl, cycloalkylalkoxy, —CN, —NO₂, —OH, ═O, —OCF₃, —CF₃, NR⁴R⁵, —S(O)ₘR⁶, —S(O)₂NR⁴R⁵, —OS(O)₂R⁶, —OS(O)₂CF₃, —O(CH₂)ₙNR⁴R⁵, —C(O)CF₃, —C(O)alkyl, —C(O)cycloalkyl, —C(O)aralkyl, —C(O)Ar, —C(O)(CH₂)ₙOR⁶, C(O)(CH₂)ₙNR⁴R⁵, —C(O)alkoxy, —C(O)NR⁴R⁵, —(CH₂)ₙC(O)alkoxy, (CH₂)ₙOC(O)R⁶, —O(CH₂)ₙOR⁶, —(CH₂)ₙOR⁶, -(CH₂)ₙNR⁴R⁵ (CH₂)ₙC(O)NR⁴R⁵, —(CH₂)ₙN(R⁴)C(O)R⁶, —(CH₂)ₙS(O)₂NR⁴R⁵, (CH₂)ₙN(R⁴)S(O)₂R⁶, -ZAr, —(CH₂)ₙS(O)₂R⁶, —(OCH₂)ₙS(O)₂R⁶, N(R⁴)S(O)₂R⁶, —N(R⁴)C(O)R⁶, -(CH₂)ₙN(R⁴)S(O)₂R⁶, —(CH₂)ₙN(R⁴)C(O)R⁶ or —(CH₂)ₙC(O)alkyl;
R³ is selected from alkyl, alkoxy, —CF₃, halo, —O(CH₂)ₙOR⁶, —O(CH₂)ₙNR⁴R⁵, phenyl, cyclohexyl, benzo[1,3]dioxolyl, morpholinyl, pyridyl, pyrimidinyl, pyrazinyl, piperazinyl, piperidinyl, pyridizinyl, thienyl, furyl, pyrazolyl, pyrrolyl, triazolyl, indanyl, imidazolyl, oxazolyl, thiazolyl, oxadiazolyl, isothiazolyl, isoxazolyl or thiadiazolyl; wherein said alkyl, alkoxy, phenyl, cyclohexyl, benzo[1,3]dioxolyl, morpholinyl, pyridyl, pyrimidinyl, pyrazinyl, piperazinyl, piperidinyl, pyridizinyl, thienyl, furyl, pyrazolyl, pyrrolyl, triazolyl, indanyl, imidazolyl, oxazolyl, thiazolyl, oxdiazolyl, isothiazolyl, isoxazolyl and thiadiazolyl groups may be optionally substituted by one or more groups, which may be the same or different, selected from R²; R⁴ and R⁵ may be the same or different and represent —H or alkyl or R⁴ and R⁵ together with the nitrogen atom to which they are attached form a heterocyclic ring; R⁶ is —H, alkyl or aryl; R⁷ is —H, alkyl or aryl; R⁸ is selected from H, alkyl, hydroxyalkyl, cycloalkyl, aralkyl, alkoxyalkyl, cycloalkylalkyl, heterocyclylalkyl, —S(O)ₘR⁶—C(O)CF₃, —C(O)alkyl, —C(O)cycloalkyl, —C(O)aralkyl, —C(O)Ar, —C(O)(CH₂)ₙOR⁶, —C(O)(CH₂)ₙNR⁴R⁵, C(O)alkoxy, —C(O)NR⁴R⁵, —(CH₂)ₙC(O)alkoxy, —(CH₂)ᵣOC(O)R⁶, R(CH₂)ₙOR⁶, —(CH₂)ₙNR⁴R⁵, —(CH₂)ₙC(O)NR⁴R⁵, —(CH₂)N(R⁴)C(O)R⁶—(CH₂)ₙS(O)₂NR⁴R⁵, —(CH₂)ₙN(R⁴)S(O)₂R⁶, —(CH₂)ₙS(O)₂R⁶, —(CH₂)ₙN(R⁴)S(O)₂R⁶, —(CH₂)ₙN(R⁴)C(O)R⁶ or —(CH₂)ₙC(O)alkyl; or where X is NR⁸ and Y is C(R⁹)₂, R⁸ may combine with R¹to form a benzoquinuclidine group; R⁹ is H or R¹; Ar is aryl or heteroaryl, each of which may be optionally substituted by R₂;
Z is a bond, O, S, NR⁷ or CH₂;
m is 0, 1 or 2;
n is an integer value from 1 to 6;
q and r are independently selected from 0, 1, 2 or 3;
s is 0, 1, 2 or 3; and
X and Y are selected from the following combinations:

| X | Y |
|---|---|
| N | CR⁹ |
| NR⁸ | C(R⁹)₂ |
| CR⁹ | N |
| C(R⁹)₂ | NR₈ |

with the proviso that said compound of formula (I) is not a compound selected from: N-{3-[(N,N-Dimethylamino)methyl]-1,2,3,4-tetrahydro-7-quinolinyl}-4-biphenylcarboxamide; N-{3-[(N,N-Dimethylamino)methyl]-1-formyl-1,2,3,4-tetrahydro-7-quinolinyl}-4-biphenylcarboxamide; N-{1-Acetyl-3-[(N,N-dimethylamino)methyl]-1,2,3,4-tetrahydro-7-quinolinyl-}-4-biphenylcarboxamide; N-{3-[(N,N-Dimethylamino)methyl]-1-methylsulfonyl-1,2,3,4-tetrahydro-7-quinolinyl}-4-biphenylcarboxamide; 5-amino-N-isoquinolin-5-yl-1-[3-(trifluoromethyl)phenyl]-1H-pyrazole-3-carboxamide; 5-methyl-N-quinolin-8-yl-1-[3-(trifluoromethyl)phenyl]-1H-pyrazole-3-carboxamide; 5-methyl-N-quinolin-7-yl-1-[3-trifluoromethyl)phenyl]-1H-pyrazole-3-carboxamide; 5-methyl-N-quinolin-3-yl-1-[3-(trifluoromethyl)phenyl]-1H-pyrazole-3-carboxamide; N-isoquinolin-5-yl-5-methyl-1-[3-(trifluoromethyl)phenyl]-1H-pyrazole-3-carboxamide; 5-methyl-N-quinolin-5-yl-1-[3-(trifluoromethyl)phenyl]-1H-pyrazole-3-carboxamide; 1-(3-chlorophenyl)-N-isoquinolin-5-yl-5-methyl-1H-pyrazole-3-carboxamide; N-isoquinolin-5-yl-1-(3-methoxyphenyl)-5-methyl-1H-pyrazole-3-carboxamide; 1-(3-fluorophenyl)-N— isoquinolin-5-yl-5-methyl-1H-pyrazole-3-carboxamide; 1-(2-chloro-5-trifluoromethylphenyl)-N-isoquinolin-5-yl-5-methyl-1H-pyrazole-3-carboxamide; 5-methyl-N-(3-methylisoquinolin-5-yl)-1-[3-(trifluoromethyl)phenyl]-1H-pyrazole-3-carboxamide; and 5-methyl-N-(1,2,3,4-tetrahydroisoquinolin-5-yl)-1-[3-(trifluoromethyl)phenyl]-1H-pyrazole-3-carboxamide.

In other embodiments, the method of the present invention uses a compound selected from any of the specific compounds, groups, or genera described in U.S. Patent Publication No. 2005/0165049. Such groups, genera and specific compounds are described, for example, on pages 2-6 and 10-25 and in the claims of U.S. Patent Publication No. 2005/0165049, said portions of which are likewise incorporated by reference herein.

In another embodiment, compounds which can be used in the method of inhibiting a taste, such as hot/peppery, bitter, or sour, include compounds disclosed in U.S. Patent Publication No. 2005/0107388, which is hereby incorporated by reference in its entirety. Specifically, such compounds include a compound having the formula:

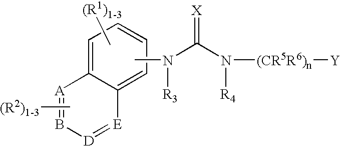

wherein A, B, D and E are each C or N with the proviso that one or more are N;
R₁ and R₂ are each independently hydrogen, halogen, hydroxy, C₁₋₆alkyl, C₂₋₆alkenyl, C₂₋₆alkynyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₅cycloalkylC₁₋₄alkyl, NR₇R₈, carboxy, esterified carboxy, C₁₋₆alkyl substituted with a group selected from NR₇R₈, carboxy and esterified carboxy, or C₁₋₆alkoxy substituted with a group selected from NR₇R₈, carboxy and esterified carboxy;
R₃ and R₄ are each independently hydrogen, C₁₋₆alkyl, C₂₋₆alkenyl or C₂₋₆alkynyl;
R₅ and R₆ are, at each occurrence, independently hydrogen, C₁₋₆alkyl, C₂₋₆alkenyl, C₂₋₆alkynyl, C₁₋₆alkoxy, C₁₋₆acyloxy, carboxy, esterified carboxy, CONR₇R₈, SO₂R₇, SO₂NR₇R₈, aryl, heteroaryl, heterocyclyl, or C₁₋₆alkyl substituted with a group selected from hydroxy, C₁₋₆alkoxy, C₁₋₆acyloxy, carboxy, esterified carboxy, NR₇R₈, CONR₇R₈, SR₇, SO₂R₇, SO₂NR₇R₈, aryl, heteroaryl and heterocyclyl; or
R₅ and R₆ and the carbon atom to which they are attached together form a carbocyclic ring of 3 to 6 carbon atoms;
R₇ and R₈ are, at each occurrence, independently hydrogen, C₁₋₆alkyl, C₂₋₆alkenyl, C₂₋₆alkynyl, C₃₋₇cycloalkyl or fluoroC₁₋₆alkyl; or
R₇ and R₈ and the nitrogen atom to which they are attached together form a heteroaliphatic ring of 4 to 7 ring atoms, optionally substituted by one or two groups selected from hydroxy or C₁₋₄alkoxy, which ring may optionally contain as one of the said ring atoms an oxygen or a sulfur atom, a group S(O) or S(O)₂, or a second nitrogen atom which will be part of a NH or NRₐ moiety where Rₐ is C₁₋₄alkyl optionally substituted by hydroxy or C₁₋₄alkoxy; X is an oxygen or sulfur atom or the group ═NCN; Y is an aryl, heteroaryl, carbocyclyl or fused-carbocyclyl group; and
n is either zero or an integer from 1 to 3;
or a pharmaceutically acceptable salt, N-oxide or a prodrug thereof.

In other embodiments, the method of the present invention uses a compound selected from any of the specific groups or genera described in U.S. Patent Publication No. 2005/0107388. Such groups, genera and specific compounds are described, for example, on pages 1-3, 5-7 and 11-36 and in the claims of U.S. Patent Publication No. 2005/0107388, said portions of which are likewise incorporated by reference herein.

In another embodiment, compounds which can be used in the method of inhibiting a taste, such as hot/peppery, bitter, or sour, include compounds disclosed in U.S. Patent Publication No. 2005/0187291, which is hereby incorporated by reference in its entirety. Specifically, such compounds include a compound having the formula:

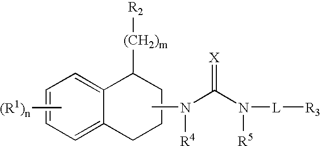

wherein R₁ is a substituent independently selected from the group consisting of hydrogen; hydroxy; fluoro; and chloro; and C₁₋₈alkanoyloxy;
n is an integer from 1 to 3;
m is an integer from 0 to 3;
R₂ is independently selected from the group consisting of hydrogen; hydroxy; C₁₋₈alkanyl; C₂₋₈alkenyl; C₁₋₈alkylidenyl; C₁₋₈alkylidenyl; fluoro; chloro; C₃₋₈cycloalkenyl; phenyl optionally substituted with one to three substituents independently selected from the group consisting of halogen, hydroxy, C₁₋₈alkanyl, C₁₋₈alkanyloxy, phenyl(Cl-g)alkanyloxy, fluorinated alkanyl, cyano, nitro, amino, C₁₋₈alkanylamino, and C₁₋₈dialkanylamino; naphthyl optionally substituted with one to three substituents independently selected from the group consisting of halogen, hydroxy, C₁₋₈alkanyl, C₁₋₈alkanyloxy, phenyl(C₁₋₈)alkanyloxy, fluorinated alkanyl, cyano, nitro, amino, C₁₋₈alkanylamino, and C₁₋₈dialkanylamino; phenoxy optionally substituted with one to three substituents independently selected from the group consisting of halogen, hydroxy, C₁₋₈alkanyl, C₁₋₈alkanyloxy, fluorinated alkanyl, cyano and nitro; and heteroaryl optionally substituted with one to three substituents independently selected from the group consisting of C₁₋₆alkanyl and halogen wherein said heteroaryl is pyridyl, pyrimidyl, furyl, thienyl or imidazolyl; pyrrolidino; and piperidino;
L is a direct bond, C₁₋₈alkandiyl, C₂₋₈alkendiyl, C₁₋₈alkyndiyl, or C₃₋₈cycloalkandiyl;
R₃ is selected from the group consisting of naphthyl substituted with hydroxyl; quinolinyl optionally substituted with one or more substituents selected from the group consisting of methyl and chloro; quinolinyl-N-oxide; isoquinolinyl optionally substituted with one or more substituents selected from the group consisting of methyl and chloro and isoquinolinyl-N-oxide;
R₄ is selected from the group consisting of hydrogen and C₁₋₈alkanyl;
R₅ is selected from the group consisting of hydrogen and C₁₋₈alkanyl;
X is selected from the group consisting of O and S; and enantiomers, diastereomers, tautomers, solvates, and pharmaceutically acceptable salts thereof.

In other embodiments, the method of the present invention uses a compound selected from any of the specific compounds, groups, or genera described in U.S. Patent Publication No. 2005/0187291. Such groups, genera, and specific compounds are described, for example, on pages 1-2, 6-25 and 31-36 and in the claims of U.S. Patent Publication No. 2005/0187291, of which portions are likewise incorporated by reference herein.

In another embodiment, compounds which can be used in the method of inhibiting a taste, such as hot/peppery, bitter, or sour, include compounds disclosed in U.S. Patent Publication No. 2005/0154230, which is hereby incorporated by reference in its entirety. Specifically, such compounds include a compound having the formula:

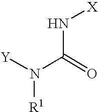

or its tautomeric or stereoisomeric form, or a or a pharmaceutically acceptable salt thereof, wherein:
Y is

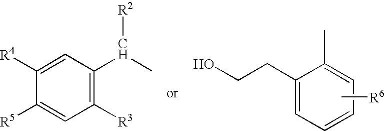

X is C₁₋₆alkyl substituted by phenyl or naphthyl (wherein said phenyl and naphthyl are optionally substituted by R¹¹, R¹² and R¹³), aryl or heterocyclic ring, wherein said aryl and heterocyclic ring are optionally substituted by R¹¹, R¹² and R¹³ and are are selected from the group consisting of phenyl, naphthyl, pyridyl, carbazolyl, fluorenyl, thienyl, pyrimidyl, benzodioxolyl, indazolyl, and quinolyl, in which R¹¹, R¹² and R¹³ independently represent hydrogen, halogen, C₁₋₆alkyl, mono-, di-, or tri-halogen substituted C₁₋₆alkyl, nitro, cyano, C₁₋₆alkoxy, hydroxy, piperidino, furyl, thienyl, benzyloxy, anilino, naphthyl, C₁₋₆alkylcarbamoyl, carbamoyl, carboxyl, amino, C₁₋₆alkylamino, di(C₁₋₆alkyl)amino, C₁₋₆alkoxycarbonyl, benzyl, phenoxy, C₁₋₆alkyl substituted phenoxy, pyridyl, halogen substituted phenoxy, C₁₋₆alkylthio, C₁₋₆alkanoyl, C₁₋₆alkanoylamino, hydroxy substituted C₁₋₆alkyl, mono-, di-, or tri-halogen substituted C₁₋₆alkyloxy, or phenyl optionally substituted by one to three substituents, in which the substituents are each different or identical and selected from the group consisting of hydrogen, halogen, C₁₋₆alkyl, C₁₋₆alkoxy, pyridyl, mono-, di-, or tri-halogen substituted C₁₋₆alkyl, nitro, cyano, benzyloxy, thienyl, C₁₋₆alkanoyl, C₁₋₆alkoxycarbonyl, C₁₋₆alkylthio, di(C₁₋₆alkyl)amino, and C₁₋₆alkylamino, mono, di, or tri halogen substituted C₁₋₆alkyloxy;
R¹is hydrogen,
R² is hydrogen,
R³ is hydrogen, or
R² and R³ together form —(CH₂)ₘ— (wherein m represents 1, 2, 3 or 4), or
R¹ and R³ together form —(CH₂)ₙ— (wherein n represents 1, 2, or 3);
R⁴ is hydrogen, halogen, C₁₋₆alkoxy, hydroxy, C₁₋₆alkoxy substituted benzyloxy, sulfamoyl, C₁₋₆alkylsulfamoyl, di(C₁₋₆alkyl)sulfamoyl, di(C₁₋₆alkyl)aminoC₁₋₆alkylene sulfamoyl, hydroxyC₁₋₆alkylpiperazinosulfonyl, C₁₋₆alkylsulfonylamino, nitro, amino, C₁₋₆alkanoylamino, C₁₋₆alkoxyC₁₋₆alkyleneoxy,
R⁵ is hydrogen, halogen, C₁₋₆alkoxy, hydroxy, C₁₋₆alkoxy substituted benzyloxy, sulfamoyl, C₁₋₆alkylsulfamoyl, di(C₁₋₆alkyl)sulfamoyl, di(C₁₋₆alkyl)amino C₁₋₆alkylenesulfamoyl, hydroxyC₁₋₆alkylpiperazinosulfonyl, C₁₋₆ alkylsulfonylamino, nitro, amino, C₁₋₆alkanoylamino, C₁₋₆alkoxyC₁₋₆ alkyleneoxy, or R⁴ and R⁵ together form —O—(CH₂)—O—; and
R⁶ is hydrogen, halogen, C₁₋₆alkyl, mono-, di-, or tri-halogen substituted C₁₋₆alkyl, nitro, cyano, C₁₋₆alkoxy, hydroxy, C₁₋₆alkylcarbamoyl, carbamoyl, carboxyl, amino, C₁₋₆alkylamino, di(C₁₋₆ alkyl)amino, C₁₋₆alkoxycarbonyl, phenyl, benzyl, phenoxy, halogen substituted phenoxy, C₁₋₆alkylthio, C₁₋₆alkanoyl, C₁₋₆alkanoylamino, hydroxy substituted C₁₋₄ alkyl, mono-, di-, or tri-halogen substituted C₁₋₆ alkoxy.

In other embodiments, the method of the present invention uses a compound selected from any of the specific compounds, groups, or genera described in U.S. Patent Publication No. 2005/0154230. Such groups, genera and specific compounds are described, for example, on pages 1-5 and 18-77 and in the claims of U.S. Patent Publication No. 2005/0154230, of which portions are likewise incorporated by reference herein.

In another embodiment, compounds which can be used in the method of inhibiting a taste, such as hot/peppery, bitter, or sour, include compounds disclosed in U.S. Patent Publication No. 2005/0113576, which is hereby incorporated by reference in its entirety. Specifically, such compounds include a compound having the formula:

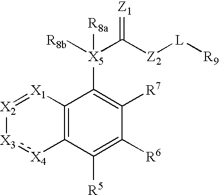

or a pharmaceutically acceptable salt or prodrug thereof, wherein: is absent or a single bond;
X¹ is selected from the group consisting of N and CR₁;
X₂ is selected from the group consisting of N and CR₂;
X₃ is selected from the group consisting of N, NR₃, and CR₃;
X⁴ is a bond or selected from the group consisting of N and CR₄;
X₅ is selected from the group consisting of N and C; provided that at least one of X₁, X₂, X₃, and X⁴ is N;
Z₁ is selected from the group consisting of O, NH, and S;
Z₂ is a bond or selected from the group consisting of NH and O; L is selected from the group consisting of alkenylene, alkylene, alkynylene, cycloalkylene,

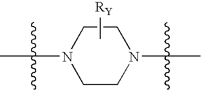

—(CH₂)ₙ—O—(CH₂)ₙ—, and N(R_{Y}), wherein the left end of —(CH₂)ₘ—O—(CH₂)ₙ— is attached to Z₂ and the right end is attached to R₉;
m and n are each independently 0-6;
R_{Y} is selected from the group consisting of hydrogen and alkyl;
R₁, R₃, R₅, R₆, and R₇ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})carbonyl, (NZ_{A}Z_{B})carbonylalkyl and (NZ_{A}Z_{B})sulfonyl, wherein Z_{A} and Z_{B} are each independently selected from the group consisting of hydrogen, alkyl, alkylcarbonyl, formyl, aryl, and arylalkyl;
R₂ and R₄ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, —NR_{A}S(O)₂R_{B}, —S(O)₂OR_{A}, —S(O)₂R_{B}, —NZ_{A}Z_{B}, (NZ_{A}Z_{B})alkyl, (NZ_{A}Z_{B})alkylcarbonyl-, (NZ_{A}Z_{B})carbonyl, (NZ_{A}Z_{B})carbonylalkyl, (NZ_{A}Z_{B})sulfonyl, (NZ_{A}Z_{B})C(═NH)—, (NZ_{A}Z_{B})C(═NCN)NH—, and (NZ_{A}Z_{B})C(═NH)NH—; R_{A} is selected from the group consisting of hydrogen and alkyl; R_{B} is selected from the group consisting of alkyl, aryl, and arylalkyl;
R₈ₐ is selected from the group consisting of hydrogen and alkyl;
R_{8b} is absent when X₅ is N or
R_{9b} is selected from the group consisting of hydrogen, alkoxy, alkoxycarbonylalkyl, alkyl, alkylcarbonyloxy, alkylsulfonyloxy, halogen, and hydroxy when X₅ is C; and R₉ is selected from the group consisting of hydrogen, aryl, cycloalkyl, and heterocycle.

In other embodiments, the method of the present invention uses a compound selected from any of the specific compounds, groups, or genera described in U.S. Patent Publication No. 2005/0113576. Such groups, genera and specific compounds are described, for example, on pages 1-9 and 23-77 and in the claims of U.S. Patent Publication No. 2005/0113576, said portions of which are likewise incorporated by reference herein.

In another embodiment, compounds which can be used in the method of inhibiting a taste, such as hot/peppery, bitter, or sour, include compounds disclosed in U.S. Patent Publication No. 2005/0049241, which is hereby incorporated by reference in its entirety. Specifically, such compounds include a compound having the formula:

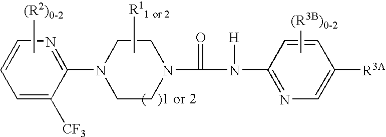

wherein,
R¹ is a substituent selected from the group consisting of —H, —C₁₋₆alkyl, —C₂₋₆alkenyl, —C₂₋₆alkynyl, —C₃₋₇cycloalkyl, perhaloC₁₋₄alkyl and —NR^{a}R^{b} (where R^{a} and R^{b} are independently —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O, —N═, >NH or >N(C₁₋₄alkyl) and optionally having one unsaturated bond in the ring), where said —C₁₋₆alkyl, —C₂₋₆alkenyl or —C₂₋₆alkynyl primary substituent is optionally mono-substituted with a substituent selected from the group consisting of halo, —C₃₋₇cycloalkyl, perhaloC₁₋₄alkyl, perhaloC₁₋₄alkoxy, hydroxy, —C₁₋₄alkoxy, —NR^{a}R^{b}, —S(O)₀₋₂C₁₋₄alkyl, —(C═O)C₁₋₄alkyl and —CONRₐR_{b}, or alternatively,
two R¹ are taken together to form a bridging group between any two nonadjacent carbon members of the piperazinylene or homopiperazinylene ring, the bridging group selected from the group consisting of —C₁₋₄alkylene-, —CH₂OCH₂—, —CH₂CH₂OCH₂—, —CH₂SCH₂—, —CH₂CH₂SCH₂—, —CH₂NHCH₂—, —CH₂CH₂NHCH₂—, —CH₂N(CH₃)CH₂— and —CH₂CH₂N(CH₃)CH₂—;
R₂ is a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with O, S, >NH or >N(C₁₋₆alkyl)), —OH, —CN, —NO₂, —N(R^{y})R^{z} (wherein R_{y} and R^{z} are independently selected from H, C₁₋₄alkyl and C₂₋₄alkenyl, or may be taken together with the nitrogen of attachment to form an otherwise aliphatic hydrocarbon ring, said ring having 4 to 7 members, optionally having one carbon replaced with >O, ═N—, >NH or >N(C₁₋₄alkyl) and optionally having one unsaturated bond in the ring), —(C═O)N(R^{y})R^{z}, —(N—R^{t})COR^{t} (wherein R^{t} is H or C₁₋₆alkyl), —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{y})R^{z}, —(C═O)N(R^{y})R^{z}, —(N—R^{t})COR^{t}, —(N—R^{t})SO₂C₁₋₆alkyl, —(C═O)C₁₋₆alkyl, —(C═O)phenyl, —(S═(O)₀₋₂)—C₁₋₆alkyl, —SO₂N(R^{y})R^{z}, —SCF₃, halo, perhaloC₁₋₄alkyl, perhaloC₁₋₄alkoxy, —COOH and —COOC₁₋₆alkyl;
R^{3A} and R^{3B} are, independently, a substituent selected from the group consisting of —C₁₋₆alkyl, —C₂₋₆alkenyl, —C₂₋₆alkynyl, phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, —N(R^{p})R^{q} (wherein R^{p} and R^{q} are independently selected from —H, —C₁₋₄alkyl and —C₂₋₄alkenyl, or may be taken together with the nitrogen of attachment to form an otherwise aliphatic hydrocarbon ring, said ring having 4 to 7 members, optionally having one carbon replaced with >O. ═N—, >NH or >N(C₁₋₄alkyl) and optionally having one unsaturated bond in the ring), —(C═O)N(R^{p})R^{q}(N—R^{s})COR^{s} (wherein R^{s} is —H or —C₁₋₆alkyl), —(N—R^{s})SO₂C₁₋₆alkyl-, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl, where said —C₁₋₆alkyl, —C₂₋₆alkenyl or —C₂₋₆alkynyl primary substituent is optionally mono-substituted with a substituent selected from the group consisting of phenyl, —OC₁₋₆alkyl, —O-phenyl, —O-benzyl, —C₃₋₇cycloalkyl, —OC₃₋₇cycloalkyl, —C₅₋₇cycloalkyl (in which a carbon member is the point of attachment and one member is replaced with >O, >S, >NH or >N(C₁₋₆alkyl)), —OH, —CN, —NO₂, N(R^{p})R^{q}, —(C═O)N(R^{p})R^{q}, (N—R^{s})COR^{s}, —(N—)SO₂C₁₋₆alkyl, —(C═O)C₁₋₆alkyl, —(C═O)phenyl, —(S═(O)₀₋₂)—C₁₋₆alkyl, —SO₂N(R^{p})R^{q}, —SCF₃, halo, perhaloC₁₋₄alkyl, perhaloC₁₋₄alkoxy, —COOH and —COOC₁₋₆alkyl; or a stereoisomer or pharmaceutically acceptable salt, ester, amide or prodrug thereof.

In other embodiments, the method of the present invention uses a compound selected from any of the specific compounds, groups, or genera described in U.S. Patent Publication No. 2005/0049241. Such groups, genera and specific compounds are described, for example, on pages 2-5 and 8 and in the claims of U.S. Patent Publication No. 2005/0049241, said portions of which are likewise incorporated by reference herein.

Other suitable compounds include the following, including analogues and derivatives thereof:

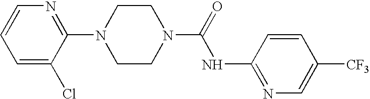

or a pharmaceutically acceptable salt thereof;

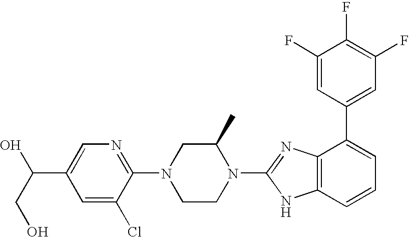

or a pharmaceutically acceptable salt thereof;

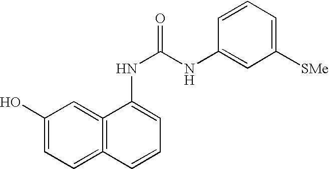

or a pharmaceutically acceptable salt thereof;

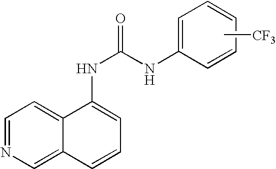

or a pharmaceutically acceptable salt thereof;

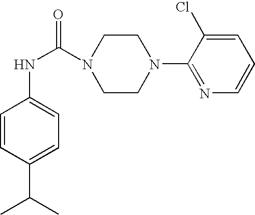

or a pharmaceutically acceptable salt thereof;

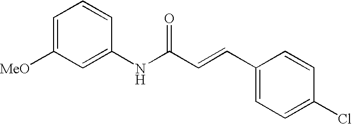

or a pharmaceutically acceptable salt thereof;

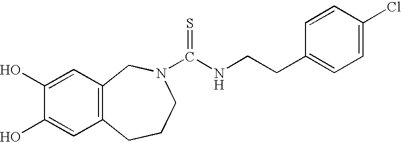

or a pharmaceutically acceptable salt thereof;

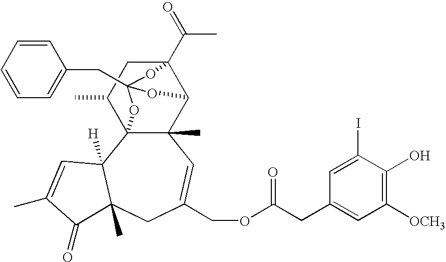

or a pharmaceutically acceptable salt thereof;

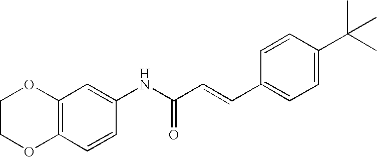

or a pharmaceutically acceptable salt thereof;

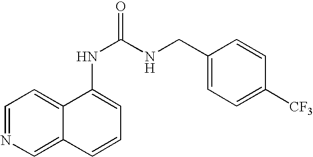

or a pharmaceutically acceptable salt thereof,

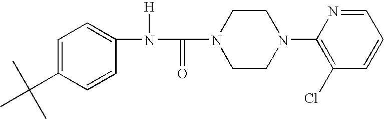

or a pharmaceutically acceptable salt thereof.

Additional examples of suitable genera, subgroups, and specific compounds useful in the methods of the present invention include those described and claimed in U.S. Pat. Nos. 7,183,430, particularly compounds disclosed at cols. 47-67; 7,183,411, particularly compounds disclosed at cols. 2-6; 7,074,805, particularly compounds disclosed at cols. 3-10; 7,074,799, particularly compounds disclosed at cols. 7-12; 7,037,927, particularly compounds disclosed at cols. 2-4; 6,984,647, particularly compounds disclosed at cols. 2-4; and 6,933,311, particularly compounds disclosed at cols. 3-15, each of which is hereby incorporated by reference in its entirety.

Additional examples of suitable genera, subgroups, and specific compounds useful in the methods of the present invention also include those described and claimed in U.S. Patent Application Publication Nos. 2006/0035882, particularly compounds in paragraphs 27-517; 2006/0035939, particularly compounds disclosed in paragraphs 43-228; 2006/0089354, particularly compounds disclosed in paragraphs 93-212; 2006/0116368, particularly compounds disclosed in paragraphs 5-34; 2006/0122394, particularly compounds disclosed in paragraphs 104-213; 2006/0142333, particularly compounds disclosed in paragraphs 10-48; 2006/0173003, particularly compounds disclosed in paragraphs 65-180; 2006/0223837, particularly compounds disclosed in paragraphs 18-35; 2006/0229308, particularly compounds disclosed in paragraphs 78-210; 2006/0258884, particularly compounds disclosed in paragraphs 7-57; 2007/0043049, particularly compounds disclosed in paragraphs 83-140; 2007/0078156, particularly compounds disclosed in paragraphs 6-135; 2007/0099896, particularly compounds disclosed in paragraphs 9-16; and 2007/0105861, particularly compounds disclosed in paragraphs 6-48, each of which is hereby incorporated by reference in its entirety.

Additional examples of suitable genera, subgroups, and specific compounds useful in the methods of the present invention also include those described and claimed in U.S. Patent Application Publication Nos. 2007/0129374, particularly compounds disclosed in paragraphs 5-27; 2007/0129423 A1, particularly compounds disclosed in paragraphs 21-26; 2007/0135423, particularly compounds disclosed in paragraphs 7-70; 2007/0135454, particularly compounds disclosed in paragraphs 9-322; 2007/0149517, particularly compounds disclosed in paragraphs 55-76; 2007/0191363, particularly compounds disclosed in paragraphs 88-112; 2007/0191374, particularly compounds disclosed in paragraphs 75-91; 2007/0197559, particularly compounds disclosed in paragraphs 91-142; 2007/0203133, particularly compounds disclosed in paragraphs 94-116; 2007/0208083, particularly compounds disclosed in paragraphs 9-25; 2007/0219203, particularly compounds disclosed in paragraphs 107-173; and 2007/0225275, particularly compounds disclosed in paragraphs 8-25, each of which is hereby incorporated by reference in its entirety.

In another embodiment, compounds useful in the methods of the present invention include 1-[3-(trifluoromethyl)pyridin-2-yl]-N-[4-(trifluoromethylsulfonyl)phenyl]-1,2,3,6-tetrahydropyridin-4-carboxamide (A-784168) and N— 1H-indazol-4-yl-N′-[(1R)-5-piperidin-1-yl-2,3-dihydro-1H-inden-1-yl]urea (A-795614). See Cui, M, et al., TRPV1 Receptors in the CNS Play a Key Role in Broad-Spectrum Analgesia of TRPV1 Antagonists, J. Neurosci. 26(37):9385-9393 (2006).

In a preferred embodiment, N-(4-tert-butyl-phenyl)-4-(3-chloropyridin-2-yl) tetrahydropyrazine-1(2H)-carboxamide (BCTC) is used in the methods and compositions of the present invention. In another embodiment, AMG9810 ((E)-3-(4-t-butylphenyl)-N-(2,3-dihydrobenzo[b][1,4]dioxin-6-yl)acrylamide) is used in the methods and compositions of the present invention.

Other compounds include SB366791 (N-(3-methoxyphenol)-4-chlorocinnamide); capsazepine; A-425619 (1-isoquinolin-5-yl-3-(4-trifluoromethylbenzyl)urea); JYL1421 and KJM429, disclosed in (Wang Y, et al., High affinity antagonists of the vanilloid receptor, Mol. Pharmacol. 62:947-956 (2002)); SB-366791 (disclosed in Gunthorpe M J, et al., Identification and characterization of SB-366791, a potent and selective vanilloid receptor (VR1/TRPV1) antagonist. Neuropharmacology 46:133-149 (2004)); N-(4-chlorobenzyl)-N-(4-hydroxy-3-iodo-5-methoxybenzyl)thiourea (Toth et al., Design of a high-affinity competitive antagonist of the vanilloid receptor selective for the calcium entrylinked receptor population. Mol. Pharmacol. 65:282-291 (2004)).

Unless otherwise indicated, the term physiologically acceptable salt refers to an acid- and/or base-addition salt of a TRPV1 antagonist. Acid-addition salts can be formed by adding an appropriate acid to the TRPV1 antagonist. Base-addition salts can be formed by adding an appropriate base to the TRPV1 antagonist. Said acid or base does not substantially degrade, decompose, or destroy said TRPV1 antagonist. Examples of suitable physiologically acceptable salts include hydrochloride, hydrobromide, acetate, furmate, maleate, oxalate, and succinate salts. Other suitable salts include sodium, potassium, carbonate, and tromethamine salts.

It is also to be understood that the present invention is considered to encompass the use of stereoisomers as well as optical isomers, e.g., mixtures of enantiomers as well as individual enantiomers and diastereomers, which arise as a consequence of structural asymmetry in selected compounds of the present series. It is further understood that the present invention encompasses the use of tautomers of a TRPV1 antagonist. Tautomers are well-known in the art and include keto-enol tautomers.

The TRPV1 antagonists used in the methods of the present invention may also be solvated, including hydrated. Hydration may occur during manufacturing of the compounds or compositions comprising the compounds, or the hydration may occur over time due to the hygroscopic nature of the compounds.

Certain TRPV1 antagonists may be derivatives referred to as “prodrugs.” The expression “prodrug” denotes a derivative of a known direct acting agent, wherein the derivative has therapeutic value that may be similar to, greater than, or less than that of the agent. Generally, the prodrug is transformed into the active agent by an enzymatic or chemical process when delivered to the subject, cell, or test media. In certain instances, prodrugs are derivatives of the compounds of the invention which have metabolically cleavable groups and become by solvolysis or under physiological conditions the compounds of the invention which are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (See Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendent on the compounds of this invention are preferred prodrugs. In some cases, it is desirable to prepare double ester type prodrugs such as (acyloxy) alkyl esters or ((alkoxycarbonyl)oxy)alkyl esters.

The terms and names used in describing the above compounds and groups of compounds are well-known in the art. (See e.g. Hawley, G. G., The Condensed Chemical Dictionary 10^{th} Edition, (1981), http://www.acdlabs.com/iuipac/nomenclature/and IUPAC, Commission on Nomenclature of Organic Chemistry, A Guide to IUPAC Nomenclature of Organic Compounds (Recommendations 1993) (1993).) It will also be understood that the definition for each group of compounds described herein will be as provided in the patent or publication originally describing that group of compounds, unless otherwise noted.

By way of example, the present invention is directed to a method of inhibiting an unpleasant taste, such as a bitter, hot/peppery, or sour taste, comprising administering to a subject in need of said inhibition an effective amount of a TRPV1 antagonist, or any of the specific subclasses and specific compounds described herein, and inhibiting the unpleasant taste by at least about 10%, 20%, 30%, 40%, 50%, 60%, 70%, 80%, 90%, or 95%, or from about 50% to about 99%. In another embodiment, the method comprises administering to a subject in need of said unpleasant taste inhibitor an effective amount of a TRPV1 antagonist, or any of the specific subclasses and specific compounds described herein, and inhibiting the unpleasant taste by about 10% to about 50%. In another embodiment, the present invention is directed to a method of inhibiting an unpleasant taste, comprising administering to a subject in need of said inhibition an effective amount of a TRPV1 antagonist, or any of the specific subclasses and specific compounds described herein, and inhibiting the unpleasant taste by at least about 10%, 20%, 30%, 40%, 50%, 60%, 70%, 80%, 90%, or 95%, or from about 50% to about 99%, or alternatively from about 10% to about 50%. In another embodiment, the present invention is directed to a method of inhibiting an unpleasant taste, comprising administering to a subject in need of said inhibition an effective amount of a TRPV1 antagonist, or any of the specific subclasses or specific compounds described herein, and inhibiting the unpleasant taste by at least about 10%, 20%, 30%, 40%, 50%, 60%, 70%, 80%, 90%, or 95%, or from about 50% to about 99%, or alternatively from about 10% to about 50%, and wherein said subject is a human.

An additional aspect of the present invention is a method of inhibiting an unpleasant taste, such as sour, bitter, or hot/peppery, of a pharmaceutical composition, comprising administering a TRPV1 antagonist, or any of the specific subgroups, subclasses, or specific compounds described above, to a subject receiving the pharmaceutical composition. The TRPV1 antagonist may be administered together with the pharmaceutical composition as separate compositions, for example either concurrently or sequentially. The TRPV1 antagonist may administered, or caused to be administered, prior to the pharmaceutical agent producing the unpleasant taste to be inhibited. Alternatively, the TRPV1 antagonist may be administered as a component of the pharmaceutical composition. In cases where the TRPV1 antagonist and the pharmaceutical agent are administered together in a single pharmaceutical composition, the pharmaceutical composition can be any type of pharmaceutical composition which is known in the art. Selected examples of such compositions are described in further detail herein.

By way of example, the method may be performed such that the unpleasant taste, such as the hot/peppery or bitter taste, being inhibited by the TRPV1 antagonist is inhibited by at least about 10%, 20%, 30%, 40%, 50%, 60%, 70%, 80%, 90%, or 95%, or from about 60% to about 99%, or alternatively from about 25% to about 50%. Thus, in a more specific embodiment, the method comprises administering a pharmaceutical composition comprising a pharmaceutically active agent, optionally one or more excipients, and one or more TRPV1 antagonists, wherein the one or more TRPV1 antagonists are present in an amount sufficient to inhibit the unpleasant taste, produced by the pharmaceutically active agent, by at least about 10%, 20%, 30%, 40%, 50%, 60%, 70%, 80%, 90%, or 95%, or from about 60% to about 99%, or alternatively from about 30% to about 60%. In another embodiment, the TRPV1 antagonist is administered in a ratio of from about 10:1 to about 1:1000, preferably from about 1:1 to about 1:100, in relation to the pharmaceutical agent.

By way of additional examples, the method of inhibiting the unpleasant taste of a pharmaceutical composition may comprise inhibiting the unpleasant taste produced by one or more agents selected from the group consisting of antipyretics, analgesics, laxatives, appetite depressants, antacidics, antiasthmatics, antidiuretics, agents active against flatulence, antimigraine agents, psychopharmacological agents, spasmolytics, sedatives, antihyperkinetics, tranquilizers, antihistamines, decongestants, beta-receptor blockers, agents for alcohol withdrawal, antitussives, fluorine supplements, local antibiotics, corticosteroid supplements, agents against goiter formation, antiepileptics, agents against dehydration, antiseptics, NSAIDs, gastrointestinal active agents, alkaloids, supplements for trace elements, ion-exchange resins, cholesterol-depressant agents, lipid-lowering agents, antiarrhythmics, and expectorants. Further specific examples of pharmaceutical compositions in accordance with the method of the invention are described below.

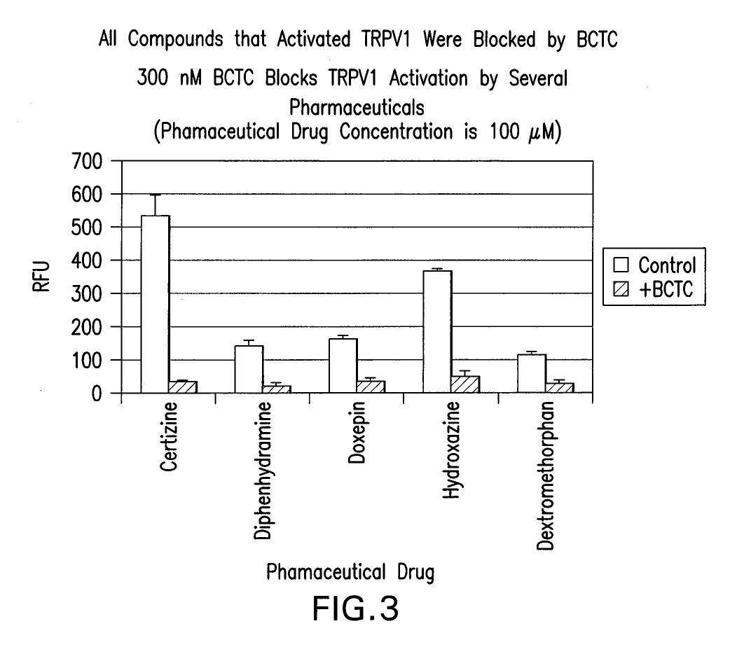

The methods described herein may be particularly suited for inhibiting an unpleasant taste of an antihistamine-containing pharmaceutical. As shown in the Examples and in FIG. 3, use of a TRPV1 antagonist blocks the activation of TRPV1 by antihistamine compounds. Accordingly, a preferred aspect of the invention is directed to inhibiting an unpleasant taste associated with an antihistamine compound by administering an effective amount of a TRPV1 antagonist. The TRPV1 may be administered in a ratio of about 1:1 to about 1:1000 with respect to the antihistamine. For example, one embodiment of the present invention may include using the TRPV1 antagonist BCTC or AMG9810 to inhibit an unpleasant taste associate with an antihistamine, such as diphenhydramine, e.g., Benadryl®.

The methods described herein may be particularly suited for inhibiting an unpleasant taste of an antiviral-containing pharmaceutical. Accordingly, a preferred aspect of the invention is directed to inhibiting an unpleasant taste associated with an antiviral compound, such as an anti-HIV drug, by administering an effective amount of a TRPV1 antagonist. The TRPV1 may be administered in a ratio of about 1:1 to about 1:1000 with respect to the antiviral drug. For example, one embodiment of the present invention may include using the TRPV1 antagonist BCTC or AMG9810 to inhibit an unpleasant taste associate with an antiviral agent.

Additionally, the method of inhibiting the unpleasant taste of a pharmaceutical composition may comprise inhibiting the unpleasant taste produced by a counterterrorism pharmaceutical. Because of the increased risk of terrorist attacks, such as chemical, nuclear, or biological attacks, the use of counterterrorism pharmaceutical agents is expected to increase in the future. A counterterrorism pharmaceutical agent includes those pharmaceutical agents that are useful in counteracting agents that can be used in a terrorist attack. Agents that have been used in terrorist acts, or considered as useful for carrying out future terrorist acts, include ricin, sarin, radioactive agents and materials, and anthrax. Pharmaceutical agents that counteract these agents are useful as a counterterrorism pharmaceutical. Such counterterrorism pharmaceuticals include, but are not limited to, antibiotics such as ciprofloxacin and doxycycline; potassium iodide; and antiviral agents. Thus, in one embodiment of the present invention, the method may be performed such that the unpleasant taste of a counterterrorism pharmaceutical, such as an antibiotic such as ciprofloxacin and doxycycline; potassium iodide; or an antiviral agent, is inhibited by the TRPV1 antagonist by at least about 10%, 20%, 30%, 40%, 50%, 60%, 70%, 80%, 90%, or 95%, or from about 60% to about 99%, or alternatively from about 25% to about 50%. In another embodiment, the TRPV1 antagonist is administered in a ratio of from about 10:1 to about 1:1000 in relation to the counterterrorism agent.

In another embodiment, a TRPV1 antagonist, or any of the specific subgroups, subclasses, or specific compounds described above, is useful for inhibiting the unpleasant taste of a nutriceutical composition. Examples of nutriceutical compositions having an unpleasant taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter and/or hot/peppery or metallic taste or aftertaste. Such amino acids include, but are not limited to, an essential amino acids selected from the group consisting of L-isomers of leucine, isoleucine, histidine, lysine, methionine, phenylalanine, threonine, tryptophan, tyrosine, and valine. Further specific examples of nutraceutical compositions in accordance with the method of the invention are described below.

By way of example, the method may be performed such that the unpleasant taste being inhibited by the TRPV1 antagonist is inhibited by at least about 10%, 20%, 30%, 40%, 50%, 60%, 70%, 80%, 90%, or 95%, or from about 60% to about 99%, or alternatively from about 20% to about 50% Thus, in a more specific embodiment, the method comprises administering a nutraceutical composition comprising a nutraceutical agent, optionally one or more excipients, and one or more TRPV1 antagonists, wherein the one or more TRPV1 antagonists are present in an amount sufficient to inhibit the unpleasant taste, produced by the nutraceutical agent, by at least about 10%, 20%, 30%, 40%, 50%, 60%, 70%, 80%, 90%, or 95%, or from about 60% to about 99%, or alternatively from about 10% to about 50%.

Inhibition of unpleasant taste in mice can be measured as outlined in Example 4 described below. Additionally, known psychophysical taste assays may be used to determine the percent inhibition of the aversive taste. For example, U.S. Pat. No. 6,942,874 at col. 10, herein incorporated by reference, discloses an in vitro taste assay that be used to identify the percent inhibition of unpleasant taste in human subjects. Briefly, a concentration of a known bitter agent is given a rating on a bitterness scale of 0 to 10, where 0 is no bitterness and 10 is the most intense bitterness the subject has ever encountered. This known bitter agent is then made up in a solution with a TRPV1 antagonist, and the subject rates the bitterness of this solution on the same scale. The ratings can be converted to % of bitter taste inhibition.

In each of the embodiments described herein, a TRPV1 antagonist, or any of the specific subgroups, subclasses, or specific compounds described above, may be used in varying ratios to the agent that is believed to cause the unpleasant taste, such as a bitter and/or hot/peppery or sour taste. For example, the method of the invention may use a composition comprised of a TRPV1 antagonist in a molar ratio of about 10:1 to about 1:1000, or alternatively administered in a molar ratio of about 5:1, about 2:1, about 1:1, about 1:10, about 1:200, or about 1:500, relative to the agent that is believed to cause the unpleasant taste, such as a bitter and/or hot/peppery or sour taste. In another example, the method of the present invention may use food product comprising one or more food ingredients and a TRPV1 antagonist, wherein the molar ratio of the TRPV1 antagonist to the food agent that causes, or is believed to cause, a bitter and/or hot/peppery taste about 10:1 to about 1:1000, or alternatively administered in a molar or weight ratio of about 5:1, about 2:1, about 1:1, about 1:10, about 1:200, or about 1:500. As will be appreciated, the various ranges and amounts of the TRPV1 antagonist can be used, with modifications if preferred, in each of the embodiments described herein.

Any amount of the TRPV1 antagonist that provides the desired degree of inhibition can be used. For example, a TRPV1 antagonist may be used at a concentration of about 0.1 μM to about 1,000 μM to inhibit an unpleasant taste. Alternatively, concentrations of about 1 μM, 50 μM, or 100 KIM of a TRPV1 antagonist may be used to inhibit the unpleasant taste.

Factors such as pharmacokinetics and pharmacodynamics of the particular compound may require that a larger or smaller amount of the TRPV1 antagonist, or any of the specific subgroups, subclasses, or specific compounds described above, be used when inhibiting an unpleasant taste in a subject in need of said inhibition. Accordingly, another embodiment includes a method of inhibiting an unpleasant taste, comprising administering to a subject in need of said inhibition, an effective amount of a TRPV1 antagonist, or any of the specific subgroups, subclasses, or specific compounds described above. In another embodiment, the method comprises administering a TRPV1 antagonist, or any of the specific subgroups, subclasses, or specific compounds described above, to a subject in an amount sufficient to inhibit TRPV1, wherein said subject has or expresses said TRPV1.

The methods of the present invention may use compositions administered in any form suitable to achieve their intended purpose. For example, the method is one which allows for the composition be administered buccally or orally. Alternatively, the method is one which allows for the composition to be administered through either an oral or nasal spray.

A TRPV1 antagonist may be incorporated into medical and/or dental compositions. Certain compositions used in diagnostic procedures have an unpleasant taste, such as contrast materials and local oral anesthetics. The inhibitors of the invention may be used to improve the comfort of subjects undergoing such procedures by improving the taste of compositions. In addition, the inhibitors of the invention may be incorporated into pharmaceutical compositions, including tablets and liquids, to improve their flavor and improve patient compliance particularly where the patient is a child or a non-human animal).

In another embodiment, a TRPV1 antagonist, or any of the specific subgroups, subclasses, or specific compounds described above, is used to inhibit the unpleasant taste of a cosmetic product. For example, but not by way of limitation, a TRPV1 antagonist may be incorporated into face creams, lipsticks, lipgloss, and the like. Also, a TRPV1 antagonist, or any of the specific subgroups, subclasses, or specific compounds described above, can be used to inhibit the unpleasant taste of lipbalm, such as Chapstick® or Burt's Beeswax® Lip Balm.

In addition, a TRPV1 antagonist, or any of the specific subgroups, subclasses, or specific compounds described above, may be incorporated into compositions that are not traditional foods, pharmaceuticals, or cosmetics, but which may contact taste membranes. Examples include, but are not limited to, soaps, shampoos, toothpaste, denture adhesive, and glue on the surfaces of stamps and envelopes. Thus, the present invention also covers a process of preparing a composition that is not a traditional food, pharmaceutical, or cosmetic, but which may contact taste membranes, according to conventional methods, wherein the improvement comprises adding a TRPV1 antagonist to said composition.

In another embodiment, a TRPV1 antagonist, or any of the specific subgroups, subclasses, or specific compounds described above, is used to inhibit a bitter and/or hot/peppery taste associated with one or more the following: bitter and/or hot/peppery pharmaceutical alkaloids such as acetaminophen, ampicillin, chlorpheniramine, chlarithromycin, doxylamine, guaifenesin, ibuprofen, pseudoephidrine hydrochloride, and ranitidine, bitter and/or hot/peppery pharmaceutical metallic salts such as zinc containing bioadhesives (denture adhesive), bitter and/or hot/peppery vitamins, bitter and/or hot/peppery components of foods such as creatine, limonin, naringin, quinizolate, and bitter and/or hot/peppery components of beverages such as caffeine, and humulone. In one embodiment, the concentration of the TRPV1 antagonist used is in the range of 0.01 mM to 20 mM and may vary depending on the amount of bitter and/or hot/peppery compound used.

In another embodiment, the present invention is directed to a method of inhibiting the unpleasant taste of a veterinary product, such as veterinary medicines, veterinary food products, veterinary supplements, and the like, that are administered to domesticated animals. In a preferred embodiment, a TRPV1 antagonist, or any of the specific subgroups, subclasses, or specific compounds described above, is used to inhibit the unpleasant taste of a veterinary product administered to a cat or dog.

In one embodiment, in each of the methods of inhibiting an unpleasant taste described herein, a TRPV1 antagonist, or any of the specific subgroups, subclasses, or specific compounds described above, is administered in an amount effective to inhibit said taste. As a nonlimiting example, the taste inhibiting effective amount of a TRPV1 antagonist, or any of the specific subgroups, subclasses, or specific compounds described above, administered in one embodiment is from about 0.01 to about 5.0 grams per 100 mL.

In another embodiment, the present invention is directed to a method of increasing the palatability and/or intake of food, comprising administering to a subject in need of such treatment one or more TRPV1 antagonists, or any of the specific subgroups, subclasses, or specific compounds described above, in an amount sufficient to decrease the palatability and/or intake of food. Taste modulating protein knockout mice have been shown to have diminished taste preference for sucrose, artificial sweeteners, and umami flavors and diminished taste aversion to bitter solutions. (See Zhang et al., Cell 112:293-301 (2003).) Thus, according to the present invention, a TRPV1 antagonist, or any of the specific subgroups, subclasses, or specific compounds described above, may be administered to a subject so that the palatability of food, as experienced by said subject, is decreased. Without being bound by theory, it is believed that a lower palatability of food can lead to a lower intake of food by the subject. Thus, in certain embodiments, by administering a TRPV1 antagonist, or any of the specific subgroups, subclasses, or specific compounds described above, to a subject, the subject will consume a decreased amount of food compared to the subject's food intake when not being administered a TRPV1 antagonist, or any of the specific subgroups, subclasses, or specific compounds described above. In other embodiments, by administering a TRPV1 antagonist, or any of the specific subgroups, subclasses, or specific compounds described above, to a subject, the subject will have a lower caloric intake compared to the subject's caloric intake when not being administered a TRPV1 antagonist, or any of the specific subgroups, subclasses, or specific compounds described above. In other embodiments, administering a TRPV1 antagonist, or any of the specific subgroups, subclasses, or specific compounds described above, to a subject can be a dieting means to facilitate or aid weight loss.

In each of the embodiments of methods described above, the subject of the method, unless otherwise limited to, may be any animal which is need of the particular treatment or effect of the method. Such animals include but are not limited to a cow, horse, sheep, pig, chicken, turkey, quail, cat, dog, mouse, rat, rabbit, monkey, or guinea pig. In other embodiments, the animal is a livestock animal, a domesticated animal, or an animal kept as a pet. In particular embodiments, the subject of the claimed method is a human.

Furthermore, in each of the embodiments of the methods described herein, a TRPV1 antagonist may be used in varying ratios to the agent that is believed to cause the unpleasant taste, such as a bitter and/or hot/peppery or sour taste. For example, a TRPV1 antagonist may be administered in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unpleasant taste. In another example, the present invention is directed to a method of inhibiting a bitter and/or hot/peppery taste of a pharmaceutical composition, comprising administering to a subject in need of such method a pharmaceutical composition and a TRPV1 antagonist, wherein the pharmaceutical composition comprises a pharmaceutically active agent and optionally one or more excipients, and wherein the TRPV1 antagonist is administered as either a component of the pharmaceutical composition or as a separate dosage form, and wherein the molar ratio of the TRPV1 antagonist to the pharmaceutically active agent is from about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the TRPV1 antagonist can be used, with modifications if preferred, in each of the embodiments described herein.

An additional aspect of the present invention is a method of inhibiting the depolarization of a chemosensory cell, comprising contacting the chemosensory cell with a TRPV1 antagonist, or any of the specific subgroups, subclasses, or specific compounds described above. For example, a TRPV1 antagonist may inhibit the depolarization of a taste receptor cell or other chemosensory cells by a mechanism other than, or in addition to, the mechanism of inhibiting a TRPV1 protein. In one embodiment, the method comprises contacting a taste receptor cell with a TRPV1 antagonist, or any of the specific subgroups, subclasses, or specific compounds described above, wherein said taste receptor cell can detect a bitter, or sour. In another embodiment of the present invention, the method comprises administering a TRPV1 antagonist, or any of the specific subgroups, subclasses, or specific compounds described above, to a subject in an amount sufficient to inhibit the depolarization of a taste receptor cell. Furthermore, when administered orally, the compound may be dispersed or diluted by saliva.

By way of example, the present invention is directed to a method of inhibiting the depolarization of a taste receptor cell, comprising contacting said taste receptor cell with a TRPV1 antagonist, or any of the specific subclasses and specific compounds listed above, and inhibiting the depolarization of the taste receptor cell by at least about 10%, 20%, 30%, 40%, 50%, 60%, 70%, 80%, 90%, or 95%, or from about 60% to about 99%, or alternatively from about 30% to about 75%. In another embodiment, the present invention is directed to a method of inhibiting the depolarization of a taste receptor cell, comprising contacting said protein with a TRPV1 antagonist, or any of the specific subclasses and specific compounds listed above, and inhibiting the depolarization of the taste receptor cell by at least about 10%, 20%, 30%, 40%, 50%, 60%, 70%, 80%, 90%, or 95%, or from about 50% to about 99%, or alternatively from about 20% to about 60%, and wherein said taste receptor cell is a naturally occurring taste modulating protein. In another embodiment, the present invention is directed to a method of inhibiting a taste receptor cell, comprising contacting said protein with a TRPV1 antagonist, or any of the specific subclasses or specific compounds listed above, and inhibiting the taste receptor cell by at least about 10%, 20%, 30%, 40%, 50%, 60%, 70%, 80%, 90%, or 95%, or from about 50% to about 99%, or alternatively from about 40% to about 80%, and wherein said taste receptor cell is a human taste receptor cell.

Any amount of the TRPV1 antagonist that provides the desired degree of inhibition can be used. For example, a TRPV1 antagonist may be used at a concentration of about 0.1 μM to about 1,000 μM to inhibit a taste receptor cell. Alternatively, concentrations of about 1 μM, 50 μM, or 100 μM of a TRPV1 antagonist may be used to inhibit the depolarization of a taste receptor cell.

In certain embodiments, a single dose or two to four divided daily doses, provided on a basis of about 0.001 to 100 mg per kilogram of body weight per day, preferably about 0.01 to about 25 mg/kg of body weight per day is appropriate. When inhibiting a taste receptor cell in vivo, the TRPV1 antagonist is preferably administered orally.

Another aspect of the present invention is directed to a method of inhibiting the taste of a food product, comprising administering one or more TRPV1 antagonists, or any of the specific subgroups, subclasses, or specific compounds described above, in conjunction with the administration of said food product to a subject. Examples of food products having an unpleasant taste include, but are not necessarily limited to, citrus fruits such as grapefruit, orange, and lemon; vegetables such as tomato, pimento, celery, melon, carrot, potato and asparagus; seasoning or flavoring materials, such as soy sauce and red pepper; soybean products; fish products; meats and processed meats; dairy products such as cheese; breads and cakes; and confectioneries such as candies, chewing gum and chocolate. Other examples of food products envisioned in accordance with the present invention are described below and throughout the specification.

The method may be performed such that the unpleasant taste of the food product being inhibited by the TRPV1 antagonist or any of the specific subgroups, subclasses, or specific compounds is decreased by at least about 10%, 20%, 30%, 40%, 50%, 60%, 70%, 80%, 90%, or 95%, or from about 60% to about 99%, or alternatively from about 20% to about 50%. Thus, in a more specific embodiment, the method comprises administering a food product comprising one or more food ingredients and one or more TRPV1 antagonists, wherein the one or more TRPV1 antagonists are present in an amount sufficient to inhibit a bitter and/or hot/peppery taste, produced by the food product, by at least about 10%, 20%, 30%, 40%, 50%, 60%, 70%, 80%, 90%, or 95%, or from about 60% to about 99%, or alternatively from about 30% to about 70%. Of course, in other embodiments, a taste may be inhibited to differing extents.

Any amount of the TRPV1 antagonist that provides the desired degree of taste inhibiting can be used. For example, a TRPV1 antagonist may be used at a concentration of about 0.1 μM to about 5,000 μM to inhibit a bitter and/or hot/peppery taste. Alternatively, concentrations of about 1 μM, 100 μM, or 500 μM of a TRPV1 antagonist may be used to inhibit a sour taste.

A food product may also include beverages and drinks. Examples of drinks having an unpleasant taste include, but are not limited to, juices of citrus fruits and vegetables, soybean, milk, coffee, cocoa, black tea, green tea, fermented tea, semi-fermented tea, refreshing drinks, beverages and milk. In certain embodiments, the taste inhibiting effective amount of a TRPV1 antagonist, or any of the specific subgroups, subclasses, or specific compounds described herein, e.g., BCTC or AMG9810, has a range of from about 0.001 to about 5.0 grams per 100 mL. In other embodiments, the taste inhibiting effective amount of a TRPV1 antagonist, or any of the specific subgroups, subclasses, or specific compounds described above, has a range of from about 0.5 to about 2 grams per 100 mL. Alternatively, a TRPV1 antagonist, or any of the specific subgroups, subclasses, or specific compounds described above, is administered in an amount of about 1 gram per 100 mL.

By way of example, the present invention is directed to a method of increasing the palatability of food and its uptake, comprising administering to a subject in need of such treatment an effective amount of a TRPV1 antagonist, or any of the specific subclasses and specific compounds listed above in a food product, and either decrease food uptake or palatability by at least about 10%, 20%, 30%, 40%, 50%, 60%, 70%, 80%, 90%, or 95%, or from about 50% to about 99%. In another embodiment, the method comprises administering to a subject in need of such treatment an effective amount of a TRPV1 antagonist, or any of the specific subclasses and specific compounds listed above in a food product, and decreasing either food uptake or palatability by about 10% to about 50%. In another embodiment, the present invention is directed to a method of decreasing palatability or food uptake, comprising administering to a subject in need of such treatment an effective amount of a TRPV1 antagonist, or any of the specific subclasses and specific compounds listed above, and either decreasing food uptake or palatability by at least about 10%, 20%, 30%, 40%, 50%, 60%, 70%, 80%, 90%, or 95%, or from about 50% to about 99%, or alternatively from about 10% to about 50%. In another embodiment, the present invention is directed to a method of decreasing food uptake or palatability, comprising administering to a subject in need of such treatment an effective amount of a TRPV1 antagonist, or any of the specific subclasses or specific compounds listed above, and decreasing either food uptake or palatability by at least about 10%, 20%, 30%, 40%, 50%, 60%, 70%, 80%, 90%, or 95%, or from about 50% to about 99%, or alternatively from about 10% to about 50%, and wherein said subject is a human.

Any amount of the TRPV1 antagonist that provides the desired degree of increase in palatability or food intake can be used. For example, a TRPV1 antagonist may be used at a concentration of about 0.1 KIM to about 1,000 μM to decrease palatability or food intake. Alternatively, concentrations of about 1 μM, 50 μM, or 100 μM of a TRPV1 antagonist may be used to decrease palatability or food intake.

The methods of the present invention may use compositions administered in any form suitable to achieve their intended purpose. Preferably, however, the method is one which allows for the composition be administered buccally, sublingually, or orally. Alternatively, the method is one which allows for the composition to be administered through either an oral or nasal spray.

As used herein, the phrase “inhibit a taste” and grammatical variants thereof, such as “taste inhibiting” and “inhibiting a taste,” refers to interfering with the perception of a unpleasant taste. The taste may be sensed to a lesser degree or not sensed at all by application of the present invention.

Unless otherwise indicated, the phrase “unpleasant taste,” or “aversive taste,” refers to a taste which is undesired by the subject. Exemplary examples of unpleasant, or aversive; tastes may include, but are not necessarily limited to bitter, sour, hot/peppery, irritating, pungent, and astringent.

### Compositions

The present invention is also directed to various compositions comprising a TRPV1 antagonist or a physiologically acceptable salt thereof for use in the methods described herein.

In one aspect, the present invention is directed to a pharmaceutical composition comprising (a) one or more active agents and (b) a TRPV1 antagonist, as defined above, including any of the specific embodiments, subclasses, or species described above, and one or more pharmaceutically acceptable carriers. Preferred compositions of the present invention are pharmaceutical compositions comprising a compound selected from one or more embodiments listed above, and one or more pharmaceutically acceptable excipients. Pharmaceutical compositions that comprise one or more TRPV1 antagonists, or any of the specific subgroups, subclasses, or specific compounds described above, may be used to formulate pharmaceutical drugs containing one or more active agents that exert a biological effect other than taste inhibition and/or inhibition of a TRPV1 protein.

Suitable active agents include pharmaceutical and biological agents that have an activity other than taste inhibition. Such active agents are well known in the art. See, e.g., The Physician's Desk Reference. Such compositions can be prepared according to procedures known in the art, for example, as described in Remington's Pharmaceutical Sciences, Mack Publishing Co., Easton, Pa., USA. In one embodiment, such an active agent includes bronchodilators, anorexiants, antihistamines, nutritional supplements, laxatives, analgesics, anesthetics, antacids, H₂-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. The pharmaceutical composition according to the present invention may comprise one or more TRPV1 antagonists, as described above, or any of the specific subgroups, subclasses, or specific compounds described above; one or more active agents that has a bitter and/or hot/peppery taste; and optionally one or more pharmaceutically acceptable carriers.

In another embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephidrine hydrochloride, pseudoephidrine sulfate, phenylpropanolamine bitartrate, and ephedrine; beta-receptor blockers, e.g., propanolol; agents for alcohol withdrawal, e.g., disulfuram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenyloine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Active substances which have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine.

By way of example, one embodiment of the present invention is directed to a pharmaceutical composition comprising (a) a pharmaceutically effective amount of an active agent; (b) BCTC or AMG9810 in an amount effective to inhibit an unpleasant taste associated with the tablet or active agent, e.g., an antihistamine; and (c) one or more pharmaceutically acceptable carriers or excipients.

In another embodiment, the composition comprises one or more TRPV1 antagonists, or any of the specific subgroups, subclasses, or specific compounds described above, and at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In another embodiment, the composition comprises one or more TRPV1 antagonists, or any of the specific subgroups, subclasses, or specific compounds described above; a biologically active agent that exhibits an activity other than taste inhibition; and at least one amino acid, such as one selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

The pharmaceutical compositions of the present invention can be in any form suitable to achieve their intended purpose. Preferably, however, the composition is one which can be administered buccally or orally. Alternatively, the pharmaceutical composition may be an oral or nasal spray.

The pharmaceutical compositions of the invention can be in any form suitable for administration to any animal that can experience the taste inhibition effects of the one or more TRPV1 antagonists, or any of the specific subgroups, subclasses, or specific compounds described above. Foremost among such animals are humans, although the invention is not intended to be so limited. Other suitable animals include canines, felines, dogs, cats, livestock, horses, cattle, sheep, and the like. A veterinary composition, as used herein, refers to a composition that is suitable for non-human animals. Such veterinary compositions are known in the art.

The pharmaceutical preparations of the present invention can be manufactured using known methods, for example, by means of conventional mixing, granulating, dragée-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Pharmaceutical excipients are well known in the art. Suitable excipients include fillers such as saccharides; binders; disintegrating agents; flow-regulating agents; and lubricants

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol. Suspensions, in addition to the active compounds, may contain suspending agents as, for example, ethoxylated isostearyl alcohols. By way of example, one embodiment of the present invention is directed to a pharmaceutical solution comprising (a) a pharmaceutically effective amount of an active agent; (b) BCTC or AMG9810 in an amount effective to inhibit an unpleasant taste associated with the solution or active agent; (c) one or more liquid carriers; and (d) one or more pharmaceutically acceptable carriers or excipients or other ingredients.

In a further embodiment, the invention is directed to a chewable tablet comprising one or more TRPV1 antagonists and one or more biologically active agents. Chewable tablets are known in the art. (See, e.g., U.S. Pat. Nos. 4,684,534 and 6,060,078, each of which is incorporated by reference in its entirety.) Any kind of medicament may be contained in the chewable tablet, preferably a medicament of bitter and/or hot/peppery taste, natural plant extracts or other organic compounds. More preferably, vitamins such as vitamin A, vitamin B, vitamin B₁, vitamin B₂, vitamin B₆, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite may be contained in the core. By way of example, one embodiment of the present invention is directed to a chewable tablet comprising (a) a pharmaceutically effective amount of an active agent; (b) BCTC or AMG9810 in an amount effective to inhibit an unpleasant taste associated with the tablet or active agent; and (c) one or more pharmaceutically acceptable carriers or excipients.

In another embodiment, the present invention is directed to an orally disintegrating composition comprising one or more active agents; one or more TRPV1 antagonists, or any of the specific subgroups, subclasses, or specific compounds described above; and optionally one or more suitable excipients or carriers. Orally disintegrating tablets are known in the art. See, e.g., U.S. Pat. Nos. 6,368,625 and 6,316,029, each of which is hereby incorporated by reference in its entirety. By way of example, one embodiment of the present invention is directed to an orally disintegrating tablet comprising (a) a pharmaceutically effective amount of an active agent; (b) BCTC or AMG9810 in an amount effective to inhibit an unpleasant taste associated with the tablet or active agent; and (c) one or more pharmaceutically acceptable carriers or excipients.

In another embodiment, the present invention is further directed to a nasal composition further comprising one or more active agents; and one or more TRPV1 antagonists, or any of the specific subgroups, subclasses, or specific compounds described above. Nasal sprays are known in the art. See, e.g., U.S. Pat. No. 6,187,332. Addition of one or more TRPV1 antagonists to a nasal spray can reduce the experience of an unpleasant taste associated with the composition of the nasal spray and/or the one or more active agents. By way of a nonlimiting example, a nasal spray composition according to the present invention comprises water (such as 95-98 weight percent), a citrate (such as 0.02 M citrate anion to 0.06 M citrate anion), a TRPV1 antagonist, and optionally phosphate (such as 0.03 M phosphate to 0.09 M phosphate). By way of additional example, one embodiment is directed to a nasal spray comprising (a) a pharmaceutically effective amount of an active agent; (b) BCTC or AMG9810 in an amount effective to inhibit an unpleasant taste associated with the tablet or active agent; and (c) one or more pharmaceutically acceptable carriers or excipients and/or other ingredients.

In another embodiment, the present invention is directed to a solid dosage form comprising a water and/or saliva activated effervescent granule, such as one having a controllable rate of effervescence, and a TRPV1 antagonist, or any of the specific subgroups, subclasses, or specific compounds described above. The effervescent composition may further comprise a pharmaceutically active compound. Effervescent pharmaceutical compositions are known in the art. (See, e.g., U.S. Pat. No. 6,649,186, which is incorporated by reference in its entirety.) The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition comprising a TRPV1 antagonist can further include one or more additional adjuvants and/or active ingredients which can be chosen from those known in the art including flavors, diluents, colors, binders, filler, surfactant, disintegrant, stabilizer, compaction vehicles, and non-effervescent disintegrants. By way of example, one embodiment of the present invention is directed to an effervescent tablet comprising (a) a pharmaceutically effective amount of an active agent; (b) BCTC or AMG9810 in an amount effective to inhibit an unpleasant taste associated with the tablet or active agent; and (c) one or more pharmaceutically acceptable carriers or excipients.

In another embodiment, the present invention is directed to a film-shaped or wafer-shaped pharmaceutical composition that comprises a TRPV1 antagonist, or any of the specific subgroups, subclasses, or specific compounds described above, and is capable of disintegrating. Such a film-shaped or wafer-shaped pharmaceutical composition can be configured, for example, as quickly disintegrating administration forms, e.g., administration forms disintegrating within a period of 1 second up to 3 minutes, or as slowly disintegrating administration forms, e.g., administration forms disintegrating within a period of 3 to 15 minutes.

The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable are polymers for use in the film-shaped or wafer-shaped pharmaceutical composition include cellulose derivatives, polyvinyl alcohol (e.g. MOWIOL™), polyacrylates, polyvinyl pyrrolidone, cellulose ethers, such as ethyl cellulose, as well as polyvinyl alcohol, polyurethane, polymethacrylates, polymethyl methacrylates and derivatives and copolymerisates of the aforementioned polymers.

In certain embodiments, the total thickness of the film-shaped or wafer-shaped pharmaceutical composition according to the invention is preferably 5 μm up to 10 mm, preferably 301m to 2 mm, and with particular preference 0.1 mm to 1 mm. The pharmaceutical preparations may round, oval, elliptic, triangular, quadrangular or polygonal shape, but they may also have any rounded shape.

In another embodiment, the present invention is directed to a composition comprising a medicament or agent contained in a coating that surrounds a gum base formulation and further comprising a unpleasant taste-inhibiting amount of a TRPV1 antagonist, or any of the specific subgroups, subclasses, or specific compounds described above. Preferably, the coating comprises at least 50% by weight of the entire product. As the center is chewed, the medicament or agent is released into the saliva. For example, U.S. Pat. No. 6,773,716, which is incorporated herein by reference in its entirety, discloses a suitable medicament or agent contained in a coating that surrounds a gum base formulation. One or more TRPV1 antagonists, or any of the specific subgroups, subclasses, or specific compounds described above, can be used in preparing the coating. Optionally, the composition may further comprise high-intensity sweeteners and appropriate flavors. It has been found that with respect to certain medicaments or agents that may have an astringent or bitter and/or hot/peppery taste that by adding a inhibiting agent to the formulation, that a much more palatable formulation, including the medicament, can be provided. In this regard, even though the medicament in, for example, its powder form may be bitter and/or hot/peppery or have an offensive taste, the matrix used as the coating of the present invention, including the inhibiting agent, will afford a product having acceptable medicinal properties. The TRPV1 antagonist, or any of the specific subgroups, subclasses, or specific compounds described above, may be present in varying amounts, such as about 30% 50%, 75%, or 90%. In another embodiment, the TRPV1 antagonist may be present in about 30% to about 99%. In other embodiments, the TRPV1 antagonist is present in about 1% to about 30%.

In yet another embodiment, the present invention is directed to a process of preparing an improved composition comprising a medicament or agent contained in a coating that surrounds a gum base formulation, wherein the improvement comprises adding a TRPV1 antagonist, or any of the specific subgroups, subclasses, or specific compounds described above, to the coating that surrounds the gum base formulation. The TRPV1 antagonist may be added in varying amounts, such as about 30% 50%, 75%, 80%, or 90%, or from about 10% to about 90%. In other embodiments, the TRPV1 antagonist is present in about 1% to about 30%.

In a further embodiment, the invention is directed to a pharmaceutical composition suitable for aerosol administration, comprising one or more active agents; a TRPV1 antagonist, or any of the specific subgroups, subclasses, or specific compounds described above; and a suitable carrier. Aerosol compositions are known in the art. (See, e.g., U.S. Pat. No. 5,011,678, which is hereby incorporated by reference in its entirety.) As a nonlimiting example, an aerosol composition according to the present invention may comprise a medically effective amount of a pharmaceutically active substance, one or more TRPV1 antagonists, or any of the specific subgroups, subclasses, or specific compounds described above, and a biocompatible propellant, such as a (hydro/fluoro)carbon propellant.

In certain embodiments, the pharmaceutical compositions of the invention comprise from about 0.001 mg to about 1000 mg of a TRPV1 antagonist, or any of the specific subgroups, subclasses, or specific compounds described above. In another embodiment, the compositions of the invention comprise from about 0.01 mg to about 10 mg of a TRPV1 antagonist, or any of the specific subgroups, subclasses, or specific compounds described above.

In another embodiment, the composition of the invention comprises a TRPV1 antagonist, or any of the specific subgroups, subclasses, or specific compounds described above, in an amount sufficient to inhibit a TRPV1 protein. By way of example, the present invention is a pharmaceutical or veterinary composition, comprising a TRPV1 antagonist, or any of the specific subclasses and specific compounds listed above, in an amount sufficient to inhibit a TRPV1 protein by at least about 10%, 20%, 30%, 40%, 50%, 60%, 70%, 80%, 90%, or 95%, or from about 50% to about 99%, or alternatively from about 10% to about 40%. In another embodiment, the present invention is a pharmaceutical or veterinary composition, comprising a TRPV1 antagonist, or any of the specific subclasses and specific compounds listed above, in an amount sufficient to inhibit a TRPV1 protein by at least about 10%, 20%, 30%, 40%, 50%, 60%, 70%, 80%, 90%, or 95%, or from about 50% to about 99%, or alternatively from about 20% to about 60%, and wherein said TRPV1 protein is a naturally occurring taste modulating protein. In another embodiment, the present invention is a pharmaceutical or veterinary composition, comprising a TRPV1 antagonist, or any of the specific subclasses and specific compounds listed above, in an amount sufficient to inhibit a TRPV1 protein by at least about 10%, 20%, 30%, 40%, 50%, 60%, 70%, 80%, 90%, or 95%, or from about 50% to about 99%, or alternatively from about 20% to about 40%, and wherein said TRPV1 protein is a naturally occurring human taste modulating protein. By way of example, one embodiment of the present invention is directed to a pharmaceutical composition comprising (a) a pharmaceutically effective amount of an active agent; (b) BCTC or AMG9810 in an amount effective to inhibit a TRPV1 protein by at least about 50%; and (c) one or more pharmaceutically acceptable carriers or excipients.

In another embodiment, the present invention is directed to a nutriceutical composition comprising one or more nutriceutical, one or more TRPV1 antagonists, or any of the specific subgroups, subclasses, or specific compounds described above, and optionally one or more carriers. Examples of nutriceutical compositions having an unpleasant taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter and/or hot/peppery or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine. Additionally, the invention is directed to a process of preparing an improved nutriceutical composition, wherein the improvement comprises adding one or more TRPV1 antagonists, or any of the specific subgroups, subclasses, or specific compounds described above, to a nutriceutical composition in an amount sufficient to inhibit a taste. In certain embodiments, one or more TRPV1 antagonists, or any of the specific subgroups, subclasses, or specific compounds described above, are added to a nutriceutical composition in an amount of about 1% to about 50%, or about 5%, 10%, or 15%, by weight.

By way of example, one embodiment of the present invention is directed to a nutriceutical composition comprising (a) an effective amount of one or more nutraceuticals; (b) BCTC or AMG9810 in an amount effective to inhibit an unpleasant taste associated with the composition or the one or more nutraceuticals; and (c) optionally one or more acceptable carriers or excipients.

In another embodiment, the present invention is directed to a dental hygienic composition comprising one or more TRPV1 antagonists, or any of the specific subgroups, subclasses, or specific compounds described above. Dental hygienic compositions are known in the art and include but are not necessarily limited to toothpaste, mouthwash, plaque rinse, dental floss, dental pain relievers (such as Anbesol™), and the like. For example, the invention includes a dental bleaching composition which comprises one or more TRPV1 antagonists, or any of the specific subgroups, subclasses, or specific compounds described above, in an amount sufficient to inhibit a bitter and/or hot/peppery taste. Dental bleaching compositions are known in the art. See, e.g., U.S. Pat. No. 6,485,709, which is herein incorporated by reference in its entirety. A dental bleaching composition of the present invention intended for use with dental trays may utilize a sticky carrier formed from a fluid and a thickener. The sticky carrier accordingly may comprise finely divided silica, such as silica fume, dispersed in a liquid, such as a polyol. Examples of suitable polyols include propylene glycol, glycerin, polypropylene glycols, sorbitol, polyethylene glycols and the like. While the carrier preferably includes thickeners, the carrier may also be only a liquid such as water or any of the liquid polyols without any thickeners.

Additionally, the invention is directed to a process of preparing an improved dental hygienic composition, wherein the improvement comprises adding one or more TRPV1 antagonists, or any of the specific subgroups, subclasses, or specific compounds described above, to a dental bleaching composition in an amount sufficient to inhibit a taste. In certain embodiments, one or more TRPV1 antagonists are added to a dental hygienic composition in an amount of about 1% to about 20%, preferably about 1% to about 5%, or about 5%, 10%, or 15%, by weight.

In another embodiment, the present invention is directed to a cosmetic product comprising one or more TRPV1 antagonists, or any of the specific subgroups, subclasses, or specific compounds described above. For example, but not by way of limitation, the cosmetic product comprising a TRPV1 antagonist, or any of the specific subgroups, subclasses, or specific compounds described above, may be a face cream, lipstick, lipgloss, and the like. Other suitable compositions of the invention include lipbalm, such as Chapstick® or Burt's Beeswax(& Lip Balm, further comprising one or more TRPV1 antagonists, or any of the specific subgroups, subclasses, or specific compounds described above.

By way of example, one embodiment of the present invention is directed to a cosmetic product comprising (a) BCTC or AMG9810 in an amount effective to inhibit an unpleasant taste associated with the cosmetic product; and (b) optionally one or more acceptable carriers or excipients.

Additionally, the invention is directed to a process of preparing an improved cosmetic product, wherein the improvement comprises adding one or more TRPV1 antagonists, or any of the specific subgroups, subclasses, or specific compounds described above, to a cosmetic product in an amount sufficient to inhibit a taste. In certain embodiments, one or more TRPV1 antagonists, or any of the specific subgroups, subclasses, or specific compounds described above, are added to a cosmetic product in an amount of about 1% to about 20%, preferably about 1% to about 5%, or about 1%, 2%, or 3%, by weight.

In another aspect, the present invention is directed to a food product comprising one or more TRPV1 antagonists, or any of the specific subgroups, subclasses, or specific compounds described above. Preferably, the food product is one which exhibits an unpleasant taste, such as a bitter and/or hot/peppery taste, which can be inhibited by a TRPV1 antagonist, or any of the specific subgroups, subclasses, or specific compounds described above. Furthermore, in a preferred embodiment, the food product comprises a TRPV1 antagonist, or any of the specific subgroups, subclasses, or specific compounds described above in an amount sufficient to inhibit an unpleasant taste. By way of example, one embodiment of the present invention is directed to a food product comprising (a) one or more conventional food ingredients; (b) BCTC or AMG9810 in an amount effective to inhibit an unpleasant taste associated with the food product.

Specific food products and food ingredients to which one of more TRPV1 antagonists, or any of the specific subgroups, subclasses, or specific compounds described above, can be added include but are not necessarily limited to, potassium chloride, ammonium chloride, sodium chloride (e.g., table salt), magnesium chloride, halide salts, naringin, caffeine, urea, magnesium sulfate, saccharin, acetosulfames, aspirin, potassium benzoate, potassium bicarbonate, potassium carbonate, potassium nitrate, potassium nitrite, potassium sulfate, potassium sulfite, potassium glutamate, food preservatives in their physiologically acceptable salts, antibiotics, unsweetened chocolate, cocoa beans, yoghurt, preservatives, flavor enhancers, dietary supplements, gelling agents, pH control agents, nutrients, processing aids, bodying agents, dispersing agents, stabilizers, colorings, coloring diluents, anticaking agents, antimicrobial agents, formulation aids, leavening agents, surface active agents, anticaking agents, nutrient supplements, alkali, acids, sequestrants, denuding agents, general purpose buffers, thickeners, cooked out juice retention agents, color fixatives in meat and meat products, color fixatives in poultry and poultry products, dough conditioners, maturing agents, yeast foods, mold retardants, emulsifiers, texturizers, binders, water correctives, miscellaneous and general purpose food additives, tableting aids, lye peeling agents, washing water agents, oxidizers, antioxidants, enzymes, extenders, fungicides, cake mixes, coffee, tea, dry mixes, non-dairy creamers, salts, animal glue adjuvant, cheese, nuts, meat and meat products, poultry and poultry product, pork and pork products, fish and fish products, vegetable and vegetable products, fruit and fruit products, smoked products such as meat, cheese fish, poultry, and vegetables, whipping agents, masticatory substances in chewing gums, dough strengtheners, animal feed, poultry feed, fish feed, pork feed, defoaming agents, juices, liquors, substances or drinks containing alcohol, beverages including but not limited to alcoholic beverages and non-alcoholic carbonated and/or non-carbonated soft drinks, whipped toppings, bulking agents used in eatables including but not limited to starches, corn solids, polysaccharides and other polymeric carbohydrates, icings, as well as potassium-containing or metal-containing substances with unpleasant tastes and the like.

Moreover, the present invention contemplates the preparation of eatables such as breads, biscuits, pancakes, cakes, pretzels, snack foods, baked goods etc. prepared using for example potassium bicarbonate or potassium carbonate in place of the sodium salts as leavening agents in conjunction with a TRPV1 antagonist, or any of the specific subgroups, subclasses, or specific compounds described above, in an amount sufficient to eliminate one or more unpleasant tastes. The TRPV1 antagonist, or any of the specific subgroups, subclasses, or specific compounds described above, can be typically present in an amount ranging from about 0.001% to about 50% by weight, preferably about 0.1% to about 10% by weight, or alternatively, from 0.1% to about 1% by weight, of the material with the unpleasant taste. The present invention also contemplates the preparation of preservatives for eatables comprising the potassium salts of benzoate, nitrate, nitrite, sulfate, and sulfite and so on, in conjunction with an appropriate concentration of a TRPV1 antagonist, or any of the specific subgroups, subclasses, or specific compounds described above, to eliminate unpleasant tastes in foodstuffs. Thus, the invention is directed to a process of preparing an improved food product, wherein the improvement comprises adding one or more TRPV1 antagonists, or any of the specific subgroups, subclasses, or specific compounds described above, to a food product. In certain embodiments, the one or more TRPV1 antagonists, or any of the specific subgroups, subclasses, or specific compounds described above, are added to a food product in an amount of about 1% to about 20%, preferably about 1% to about 5%, about 1%, 3%, or 4%, by weight.

In another embodiment, the present invention is directed to a table condiment for use on food comprising an taste-inhibiting effective amount of one or more TRPV1 antagonists. The food condiment can be used to add to a food product or meal in a similar manner as table salt or table pepper is used. The food condiment of the present invention can be used to reduce the bitter, sour, or hot/peppery taste of the food to be consumed. Such a food condiment is particularly useful in a setting where a person is consuming a meal prepared by someone else, for example, at a restaurant. In a restaurant, for example, a person can order a meal but may be discouraged from ordering a particular meal for fear that the meal will be too hot/peppery. If a meal is too hot/peppery for the person, then the person can add the food condiment of the present invention to reduce the hot/peppery taste of the meal.

In other embodiments, a food product comprises an effective amount of a TRPV1 antagonist in an amount or configuration such that the bitter, sour, or hot/peppery taste of the food product is sensed by the user for a short period of time. In certain food products, it is desirable for the consumer to experience a bitter, sour, or hot/peppery taste for a short period of time after consuming the food. The desired effect is that the particular taste is experienced quickly after chewing the food product but that the taste, e.g., bitter or hot/peppery, does not linger for too long. The present invention provides a food product which accomplishes this desired effect.

In one embodiment, the food product is made such that a first component is on the outside of the food product. The first component contains most or all of the particular taste ingredient, e.g., bitter, sour or hot/peppery. The first component can be a separate layer of food product, such as a hardened candied layer. Alternatively, the first component can be a film or coating layer containing the taste ingredient. The food product further comprises a second component, which can be a second layer or a core. The second food component contains, among other ingredients, a TRPV1 antagonist in an amount effective to inhibit the taste from the first food component.

Thus, with this embodiment, a consumer can consume the food product and experience a burst of taste, e.g., bitter, sour, or hot/peppery. This taste will be generated by the outer layer of the food product. Upon further consumption, e.g., further chewing or sucking, the effective amount of the TRPV1 antagonist is released in the oral cavity. This effective amount of the TPRV1 antagonist then inhibits the particular taste, thereby providing the consumer with a sensation of a quick burst of a particular taste without the lingering taste.

By way of example, one embodiment of the present invention is directed to a food product, e.g., chewing gum, comprising (a) a gum base comprising an effective amount of a TRPV1 antagonist, e.g., BCTC or AMG9810 in an amount effective to inhibit a hot/peppery taste; and (b) a film layer containing an amount of a hot/peppery tastant, e.g., capsaicin.

In another embodiment, the present invention is directed to an animal food product comprising one or more TRPV1 antagonists, or any of the specific subgroups, subclasses, or specific compounds described above. The one or more compounds are preferably in an amount sufficient to inhibit one or more unpleasant tastes associated with the animal food product. Animal food products are well known in the art, see, e.g., U.S. Pat. No. 6,403,142, and include dog food, cat food, rabbit food, and the like. The animal food product may also be food products useful for feeding livestock, such as cattle, bison, pigs, chicken, and the like. In another embodiment, the animal food composition of the present invention is a solid hypoallergenic pet food comprising a component that contains protein or protein fragments wherein all of said component is partially hydrolyzed and further comprises one or more TRPV1 antagonists, or any of the specific subgroups, subclasses, or specific compounds described above.

By way of example, one embodiment of the present invention is directed to an animal food product comprising (a) one or more conventional animal food ingredients; (b) BCTC or AMG9810 in an amount effective to inhibit an unpleasant taste associated with the food product.

Additionally, the invention is directed to a process of preparing an improved animal food product, wherein the improvement comprises adding one or more TRPV1 antagonists, or any of the specific subgroups, subclasses, or specific compounds described above, to an animal food product. In certain embodiments, one or more TRPV1 antagonists, or any of the specific subgroups, subclasses, or specific compounds described above, are added to an animal food product in an amount of about 1% to about 25%, about 1% to about 10%, or about 5%, 10%, or 15%, by weight.

In further embodiments of the present invention, any of the compositions described herein and containing a TRPV1 antagonist may further comprise one or more additional taste masking agents. Such masking agents include but are not limited to the group consisting of sucralose; zinc gluconate; ethyl maltol; glycine; acesulfame-k; aspartame; saccharin; fructose; xylitol; malitol; isomalt; salt; spray dried licorice root; glycyrrhizin; dextrose; sodium gluconate; sucrose; glucono-delta-lactone; ethyl vanillin; and vanillin.

In another embodiment, the present invention is directed to a composition comprising a TRPV1 antagonist, or any of the specific subgroups, subclasses, or specific compounds described above, and a carrier, wherein said carrier is suitable for an assay. Such carriers may include solid carriers and/or liquid carriers. A composition suitable for an assay may, but not necessarily, be sterile. Examples of suitable carriers for assays include dimethylsulfoxide, ethanol, dichloromethane, methanol, and the like. In another embodiment, a composition comprises a TRPV1 antagonist, or any of the specific subgroups, subclasses, or specific compounds described above, and a carrier, wherein the compound is in an amount suitable for inhibiting a TRPV1 protein.

In each of the embodiments of the compositions described herein, a TRPV1 antagonist, or any of the specific subgroups, subclasses, or specific compounds described above, may be used in varying ratios to the agent that is believed to cause the unpleasant taste, such as a bitter and/or hot/peppery or sour taste. For example, a composition of the invention may comprise a TRPV1 antagonist in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unpleasant taste, such as a bitter and/or hot/peppery or sour taste. In another example, the present invention is directed to a food product comprising one or more food ingredients and a TRPV1 antagonist, wherein the molar ratio of the TRPV1 antagonist to the food agent that causes, or is believed to cause, a bitter and/or hot/peppery taste about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the TRPV1 antagonist can be used, with modifications if preferred, in each of the embodiments described herein.

An additional aspect of the present invention is directed to a method of increasing the palatability of food and its intake comprising administering one or more TRPV1 antagonists to a subject in need of such treatment.

Factors such as pharmacokinetics and pharmacodynamics of the particular compound may require that a larger or smaller amount of the TRPV1 antagonist, or any of the specific subgroups, subclasses, or specific compounds described above, be used when increasing the palatability of food and its uptake. Accordingly, another embodiment includes a method of increasing the palatability of food and its uptake, comprising administering to a subject in need of such treatment, an effective amount of a TRPV1 antagonist, or any of the specific subgroups, subclasses, or specific compounds described above to food products. In another embodiment, the method comprises administering a TRPV1 antagonist, or any of the specific subgroups, subclasses, or specific compounds described above in a food product, to a subject in an amount sufficient to inhibit TRPV1, wherein said subject has or expresses said TRPV1.

Of course, other methods and procedures known in the art may be used to prepare certain TRPV1 antagonists. The following examples are illustrative, but not limiting, of the methods by which TRPV1 antagonists may be studied for their effects on taste functions and perceptions.

### EXAMPLES

### Example 1

### Activities of Selected Compounds

A cell-based fluorescence assay was performed using HEK293 cells to determine

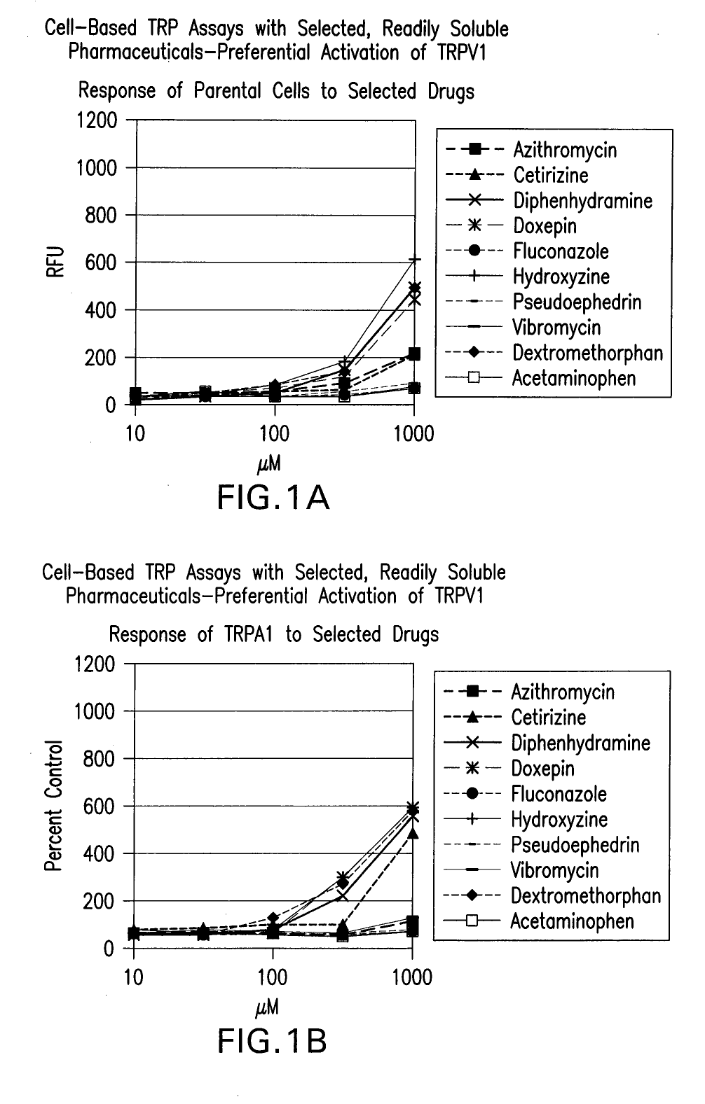

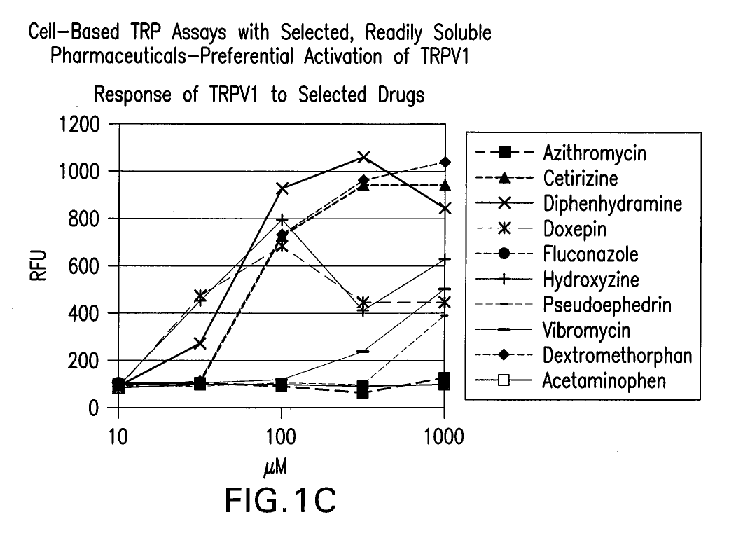

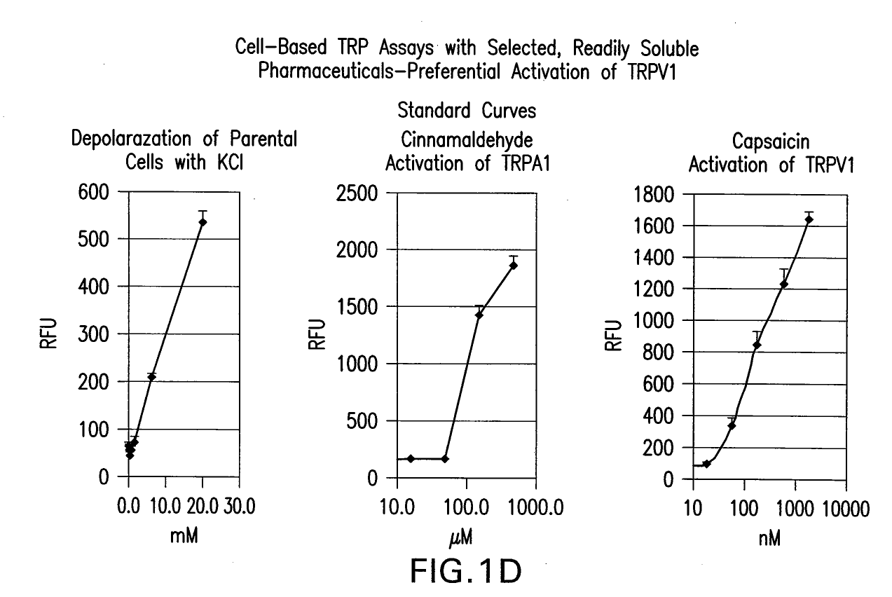

TRPV1 selectivity of readily soluble drugs. Cells either remained untransfected (Parental) in Panel A, transfected with a vector encoding human TRPA1 (TRPA1) in Panel B, or transfected with a vector encoding human TRPV1 (TRPV1) in Panel C. Parental cells and TRPA1 cells served as negative controls. All cells were incubated with 100 μM of readily soluble drugs. Cell membrane depolarization was measured in relative fluorescence units (RFU) through a coupled fluorescence emission scheme. FIG. 1. shows cetirizine, diphenhydramine, doxepin, hydroxyzine, and dextromethorphan eliciting greater membrane depolarization in TRPV1 cells as measured in RFU, compared to the results observed in Parental and TRPA1 cells. Panel D shows the response of known agonists on 3 TRP channels.

### Example 2

### Activity of Selected TRPV1 Antagonist in HEK293 Cells

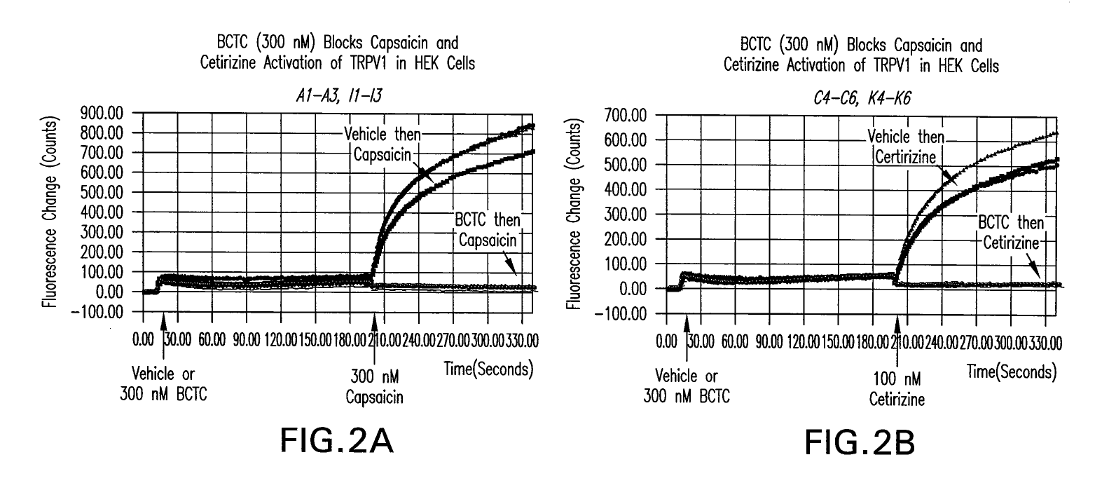

The activity of a TRPV1 antagonist was monitored over time in a another cell-based fluorescence assay. HEK293 cells were transfected with a vector encoding human TRPV1. Using the FLIPR system, the cellular membrane potential coupled to fluorescence emissions is measured in RFU over time. Panel A of FIG. 2 shows that in the first 180 seconds, no significant change in measured fluorescence occurs. Therefore no significant membrane depolarization occurs with the application of either the Vehicle or the TRPV1 antagonist, BCTC. When the transfected cells are pre-incubated with just the Vehicle, a fluorescence change is recorded upon capsaicin application. Therefore, capsaicin as a TRPV1 agonist opens the TRPV1 cation channel to allow for ion movement across the cell membrane and for membrane depolarization to occur. When the transfected cells are pre-incubated with 300 nM BCTC, no significant fluorescence change is recorded upon subsequence capsaicin application. These observations indicate that BCTC as a TRPV1 antagonist has blocked the activity of capsaicin. The results observed in Panel A of FIG. 2 for capsaicin are repeated in Panel B using Cetirizine as the TRPV1 agonist.

### Example 3

### Activity of Selected TRPV1 Antagonist in HEK293 Cells

A cell-based fluorescence assay was performed using HEK293 cells to observe inhibition of selected, readily soluble drugs by a known TRPV1 antagonist (FIG. 3). Cells were transfected with a vector encoding human TRPV1. Subsequently, the transfected cells were incubated with 100 μM of cetirizine, diphenhydramine, doxepin, hydroxyzine or dextromethorphan. As in Example 1, these compounds produce cell membrane depolarization observed through a coupled fluorescence reaction. The measured cellular response in RFU to these compounds is used as a baseline for determining the effects of TRPV1 antagonists. Upon application of 300 nM of an identified TRPV1 antagonist, BCTC (+BCTC), the total RFU counts drop significantly with respect to the baseline cellular response. The observations in FIG. 2 indicate that the TRPV1 antagonist, BCTC, is capable of blocking cell membrane depolarization produced by the application of TRPV1 activators.

### Example 4

### Behavioral Response to TRPV1 Antagonist Activity in Mice

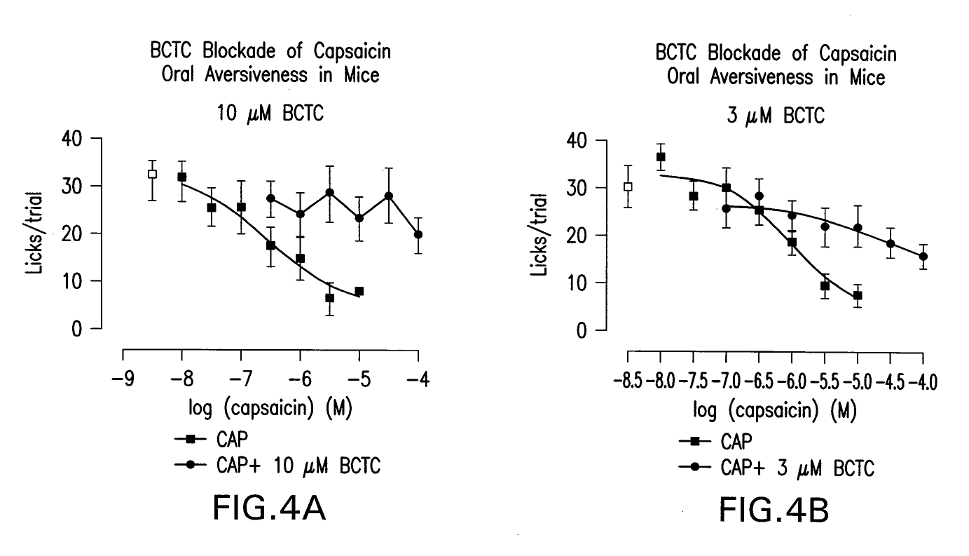

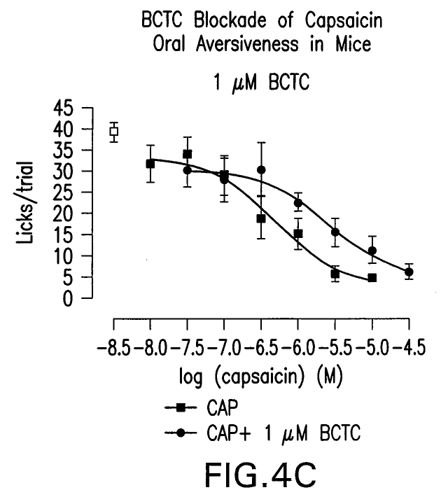

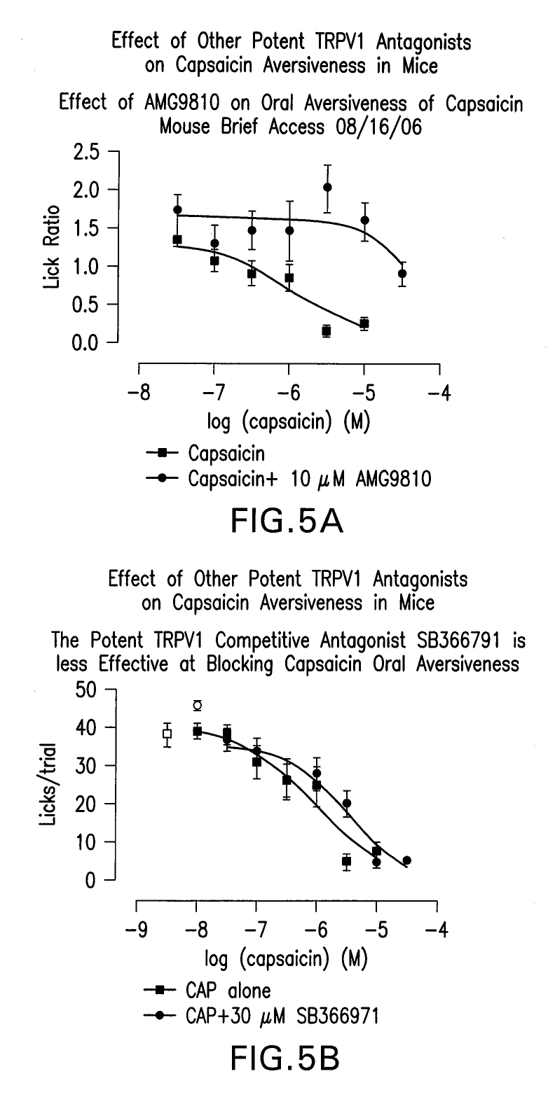

Behavioral responses in mice were studied to determine the effect of TRPV1 Antagonists in oral compositions containing capsaicin. Oral aversiveness was measured in the number of licks from a solution source recorded for a mouse per a given trial. When mice were exposed to increasing capsaicin (CAP) doses in Panels A, B and C of FIG. 4 (black squares), they licked the solution source less frequently. With increasing concentrations of BCTC, a TRPV1 antagonist being added to the solution source in combination with capsaicin, oral aversiveness to capsaicin was diminished (CAP+LG140995; black circles) in Panels A, B and C or FIG. 4. FIG. 5 extends the observations with other potent TRPV1 antagonists. AMG9810 blocks the aversiveness of capsaicin in mice, while SB366971 has little effect on capsaicin aversiveness.

Having now fully described the invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

What is claimed is:

1. A method of inhibiting one or more unpleasant tastes, comprising administering to a subject in need of said inhibition an effective amount of one or more TRPV1 antagonists, wherein said one or more tastes are selected from the group consisting of bitter, sour, hot/peppery, irritating, pungent, and astringent.

2. The method according to claim 1, wherein the taste is a hot/peppery taste.

3. The method according to claim 1, wherein the TRPV1 antagonist is administered in an amount from about 0.01 mg to about 100 mg.

4. The method according to claim 1, wherein the TRPV1 antagonist has a half-life of less than about 1 hour in a pH of about 4 or less.

5. The method according to claim 1, wherein the TRPV1 antagonist has a Ki of less than 1 micromolar.

6. The method according to claim 1, wherein the TRPV1 antagonist has a systemic bioavailability of less than about 20%, 10%, 5%, 3%, or 1%.

7. The method according to claim 1, wherein the TRPV1 antagonist has a half-life of less than about 1 hour in a pH of about 4 or less; a Ki of less than 1 micromolar; and a systemic bioavailability of less than about 20% 10%, 5%, 3%, or 1%.

8. The method according to claim 1, wherein the TRPV1 antagonist is BCTC.

9. The method according to claim 1, wherein the TRPV1 antagonist is administered as a pharmaceutical composition.

10. The method according to claim 9, wherein the TRPV1 antagonist is present in the pharmaceutical composition in an amount from about 0.001% to 50% by weight.

11. The method according to claim 1, wherein the subject is human.

12. The method according to claim 1, wherein the TRPV1 antagonist is administered as a food composition.

13. The method according to claim 12, wherein the TRPV1 antagonist is present in the food composition in an amount from about 0.00001% to 10% by weight.

14. The method according to claim 1, wherein the unpleasant taste is produced by a biologically active agent.

15. The method according to claim 1, wherein the unpleasant taste is produced by one or more agents selected from the group consisting of antipyretics, analgesics, laxatives, appetite depressants, antacidics, antiasthmatics, antidiuretics, agents active against flatulence, antimigraine agents, psychopharmacological agents, spasmolytics, sedatives, antihyperkinetics, tranquilizers, antihistaminics, decongestants, beta-receptor blockers, agents for alcohol withdrawal, antitussives, fluorine supplements, local antibiotics, corticosteroid supplements, agents against goiter formation, antiepileptics, agents against dehydration, antiseptics, NSAIDs, gastrointestinal active agents, alkaloids, supplements for trace elements, ion-exchange resins, cholesterol-depressant agents, lipid-lowering agents, antiarrhythmics, and expectorants.

16. The method according to claim 1, wherein the unpleasant taste is a hot/peppery taste.

17. A method of inhibiting the taste of a pharmaceutical comprising administering one or more TRPV1 antagonists, or a physiologically acceptable salt thereof, in conjunction with the administration of said pharmaceutical to a subject.

18. The method according to claim 17, wherein said subject is human.

19. The method according to claim 17, wherein the TRPV1 antagonist is administered in an amount from about 0.01 mg to about 100 mg.

20. A pharmaceutical composition comprising one or more active agents; one or more TRPV1 antagonists; and one or more pharmaceutically acceptable carriers or excipients;
wherein the TRPV1 antagonist is in an amount sufficient to inhibit a taste.

21. The pharmaceutical composition according to claim 19, wherein the composition is an orally disintegrating tablet formulation.

22. A food product comprising one or more TRPV1 antagonists wherein the TRPV1 antagonist is in an amount sufficient to inhibit a taste.

23. A cosmetic product comprising one or more TRPV1 antagonists wherein the TRPV1 antagonist is in an amount sufficient to inhibit a taste.
